SEQUENCE LISTING (16 sequences)

SEQ ID NO: 1 - DNA, 1557 bp, Escherichia coli
atgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctga

SEQ ID NO: 2 - DNA, 43 bp, Artificial
  primer
gcagagctctaaggaggttacaatgatcccggacgtatcacag

SEQ ID NO: 3 - DNA, 37 bp, Artificial
  primer
gcgggtaccttatcaggcgtgtttttccagccacacc

SEQ ID NO: 4 - DNA, 951 bp, Escherichia coli
atgatcaagctcggcatcgtgatggaccccatcgcaaacatcaacatcaagaaagattccagttttgctatgttgctggaagcacagcgtcgtggttacgaacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaa

SEQ ID NO: 5 - DNA, 36 bp, Artificial
  primer
agcatcgtacatatgatcaagctcggcatcgtgatg

SEQ ID NO: 6 - DNA, 36 bp, Artificial
  primer
gcagaattctcattactgctgctgtaaacgtgcttc

SEQ ID NO: 7 - DNA, 2265 bp, Streptococcus agalactiae
catatgattattgaccgtctgctgcaacgctcccatagccatctgccgatcctgcaagccacctttggtctggaacgtgaatccctgcgcattcatcagccgacccagcgtgtggcccagacgccgcatccgaaaaccctgggctctcgcaactatcacccgtacatccaaacggattatagtgaaccgcagctggaactgattaccccgatcgccaaagactctcaggaagcaatccgttttctgaaagccatttcagatgttgcaggtcgctcgattaatcatgacgaatatctgtggccgctgagtatgccgccaaaagtccgtgaagaagatattcaaatcgctcagctggaagatgcgttcgaatatgactaccgcaaatatctggaaaaaacctacggcaaactgattcagtccatctcaggtattcactataacctgggcctgggtcaagaactgctgacctcgctgtttgaactgagccaggcggataacgccattgacttccagaatcaactgtatatgaaactgtctcagaattttctgcgttaccgctggctgctgacctatctgtacggcgccagtccggtggcagaagaagatttcctggaccagaaactgaacaatccggtccgttccctgcgcaactcacatctgggttatgtgaatcacaaagatattcgtatctcgtataccagcctgaaagattacgtgaacgacctggaaaatgctgttaaatctggccagctgattgcggaaaaagaattttatagcccggttcgtctgcgcggctctaaagcctgccgtaactatctggaaaaaggtatcacgtacctggaatttcgcaccttcgatctgaatccgtttagtccgattggtatcacccaggaaacggtggataccgttcacctgtttctgctggcgctgctgtggattgacagctctagtcacatcgatcaggacattaaagaagccaaccgcctgaatgatctgatcgcactgagccatccgctggaaaaactgccgaaccaggctccggtttcagatctggtcgacgcaatgcaatcggtgattcagcactttaatctgagcccgtattaccaagatctgctggaaagtgttaaacgtcagatccaatccccggaactgaccgttgcgggtcagctgctggaaatgattgaaggtctgtccctggaaaccttcggccagcgccagggtcagatctatcatgattacgcatgggaagctccgtatgcgctgaaaggctacgaaacgatggaactgagcacccaactgctgctgttcgatgttattcagaaaggtgtgaactttgaagttctggatgaacaagaccagttcctgaaactgtggcataattcccacatcgaatatgtgaaaaacggcaatatgacgtcaaaagataactacatcgttccgctggcgatggccaataaagtggttaccaagaaaattctggacgaaaaacactttccgacgccgttcggtgatgaatttaccgaccgtaaagaagcgctgaactatttttcgcaaatccaggataaaccgattgtcgtgaaaccgaaaagcacgaacttcggcctgggtatttctatctttaaaaccagtgccaatctggcatcctacgaaaaagctattgatatcgcgtttacggaagacagcgcgatcctggtcgaagaatatattgaaggcaccgaataccgtttctttgtgctggagggtgattgtatcgcggtcctgctgcgtgtggccgcaaatgttgttggtgacggtatccataccatttcccaactggtgaaactgaaaaaccagaatccgctgcgtggctatgatcaccgctcaccgctggaagttatcgaactgggtgaagtcgaacagctgatgctggaacagcaaggctacacggtgaactcgatcccgccggaaggtaccaaaattgaactgcgtcgcaactctaatatcagtacgggcggtgatagcattgacgttaccaatacgatggatccgacctataaacagctggcagctgaaatggcagaagctatgggcgcatgggtctgtggtgtggatctgattatcccgaacgctacgcaggcgtacagcaaagacaagaaaaacgcgacctgcattgaactgaactttaatccgctgatgtatatgcatacctactgtcaagaaggtccgggtcagagcatcacgccgcgcatcctggcaaaactgtttccggaactgtaatgagaattc

SEQ ID NO: 8 - DNA, 1440 bp, Acinetobacter johnsonii
catatggataccgaaacgattgcctccgccgtcctgaacgaagaacaactgtcgctggatctgattgaagcacaatacgctctgatgaacacccgcgatcagtcgaatgcgaaaagcctggtgattctggtttccggcatcgaactggccggcaaaggtgaagcagtcaaacagctgcgcgaatgggtggacccgcgttttctgtatgttaaagccgatccgccgcacctgtttaatctgaaacagccgttctggcaaccgtatacccgctttgttccggcggagggtcagattatggtctggttcggcaactggtacggtgatctgctggctaccgcgatgcacgcatcgaaaccgctggatgacacgctgtttgatgaatatgtcagcaatatgcgtgcgttcgaacaggatctgaaaaacaataacgtcgacgtgctgaaagtgtggtttgatctgtcctggaaatcactgcaaaaacgcctggatgacatggatccgtctgaagttcattggcacaaactgcatggcctggactggcgtaacaaaaaacagtacgataccctgcaaaaactgcgtacccgctttacggatgactggcagattatcgatggtgaagatgaagacctgcgcaatcacaacttcgcccaagcaattctgacggccctgcgtcattgcccggaacacgaaaagaaagcggccctgaaatggcagcaagcaccgattccggacatcctgacccagtttgaagtgccgcaagctgaagatgcgaactacaaatcagaactgaaaaaactgacgaaacaggttgctgatgcgatgcgttgtgatgaccgcaaagtggttatcgccttcgaaggcatggacgcagctggtaaaggcggtgcaattaaacgcatcgttaaaaaactggatccgcgtgaatacgaaattcataccatcgcggccccggaaaaatatgaactgcgtcgcccgtacctgtggcgcttttggtctaaactgcagagtgatgacattaccatcttcgatcgtacgtggtatggccgcgttctggtcgaacgtgtcgaaggttttgcgaccgaagtggaatggcagcgcgcctacgcagaaattaatcgttttgagaaaaacctgagctctagtcagacggtgctgattaaattctggctggctatcgataaagacgaacaagcagctcgttttaaagcccgcgaaagcaccccgcataaacgtttcaaaatcacggaagaagattggcgtaatcgcgacaaatgggatgactatctgaaagcggccgcagatatgttcgctcacaccgacacgtcctatgcgccgtggtacattatctcaaccaacgataaacagcaagctcgtatcgaagtcctgcgtgccatcctgaaacaactgaaagccgaccgtgacaccgactaatgagaattc

SEQ ID NO: 9 - protein, 518 aa, Escherichia coli
MIPDVSQALAWLEKHPQALKGIQRGLERETLRVNADGTLATTGHPEALGSALTHKWITTDFAEALLEFITPVDGDIEHMLTFMRDLHRYTARNMGDERMWPLSMPCYIAEGQDIELAQYGTSNTGRFKTLYREGLKNRYGALMQTISGVHYNFSLPMAFWQAKCGDISGADAKEKISAGYFRVIRNYYRFGWVIPYLFGASPAICSSFLQGKPTSLPFEKTECGMYYLPYATSLRLSDLGYTNKSQSNLGITFNDLYEYVAGLKQAIKTPSEEYAKIGIEKDGKRLQINSNVLQIENELYAPIRPKRVTRSGESPSDALLRGGIEYIEVRSLDINPFSPIGVDEQQVRFLDLFMVWCALADAPEMSSSELACTRVNWNRVILEGRKPGLTLGIGCETAQFPLPQVGKDLFRDLKRVAQTLDSINGGEAYQKVCDELVACFDNPDLTFSARILRSMIDTGIGGTGKAFAEAYRNLLREEPLEILREEDFVAEREASERRQQEMEAADTEPFAVWLEKHA

SEQ ID NO: 10 - protein, 316 aa, Escherichia coli [sequence omitted]

SEQ ID NO: 11 - DNA, 2253 bp, Streptococcus agalactiae
atgattatcgatcgactgttacaaagaagccactctcatctaccgattctacaagctacatttggcttagagagagaaagccttcgtattcaccaacccactcaaagggttgctcaaacaccccatcccaaaacattagggagtcgtaactatcatccttatatccaaacggattatagtgaacctcaattagaactcattacacctattgcaaaggatagccaagaagccattcgatttttgaaagctataagtgatgttgctggacgctctatcaaccacgatgaatacttatggccgctttctatgccacccaaagtgagggaagaagacatacagattgcccaattagaggatgcttttgaatatgattatcgcaaatatttggagaagacttatggaaaactaatacaatccatctctggaattcactataatctcggtttaggtcaagagttattaacttctctctttgaattgagtcaagcagataatgctattgattttcagaaccagctttacatgaaattgtctcaaaattttttacgttaccgttggttactaacttatctatacggagcgagcccagtagctgaagaagactttttagatcagaaactgaataaccctgtccggtcacttagaaatagtcacttaggttatgtgaatcataaagatattcgtatttcttatactagcttgaaggattatgtcaacgatctagaaaatgctgtaaaaagtgggcaattgattgctgagaaagaattttattcacctgttcgtctccgtggtagtaaggcctgtcgtaattatttagaaaaaggaattacatatttagagtttcgcacttttgatctcaacccatttagcccaatcggtataacgcaggaaactgttgatactgttcaccttttcttattggcgcttttgtggatagattctagtagtcatattgaccaagatatcaaggaagccaatagattaaatgaccttatagcacttagtcatccattagaaaaattacctaatcaagcaccagtttctgacttagtggatgctatgcaatctgttatccaacattttaacttatcaccttactatcaagacctattggaatctgtaaaaaggcaaattcagtcacctgaattaacggtagctggtcaacttttagaaatgattgaaggactttccttagaaacatttggacaaagacaaggacaaatttatcacgattatgcttgggaagctccctacgccttaaagggatatgaaacgatggaactttctacccaattgctactatttgacgttatccaaaaaggagtcaacttcgaagtgctggatgaacaagatcaattcctaaaattatggcacaatagtcatattgagtacgttaaaaatggtaatatgacctcaaaagataactatattgtaccgctcgctatggctaacaaagttgttacaaaaaaaatcctagatgaaaagcatttcccaactccttttggagatgaatttactgaccgtaaagaggcacttaactatttttcacaaatccaagataaaccaatcgtcgttaagccaaaatctacaaactttggtttaggcatttctatttttaaaacatcagctaacttagcttcttatgaaaaagcaattgacatcgcttttaccgaagatagtgctattcttgtggaagaatatattgagggtaccgaataccgtttcttcgtccttgagggggactgtattgctgtattgcttagggtagcagctaatgtcgttggagatggtattcatactattagtcagctagttaaacttaaaaaccaaaaccctctcagaggctatgatcatcgctctcctctagaggttattgagctcggagaagtagaacagttaatgttggaacaacaaggatacactgttaacagtatccctccagaaggaacaaaaatagaacttcgccgtaattctaatatttccactggcggtgactctatagatgtaacaaatacaatggatcctacatataaacaactagcagctgagatggcagaggctatgggggcttgggtttgtggagttgatttaattattcctaatgccactcaagcatactcaaaagataagaaaaatgctacttgcattgaactaaactttaacccattgatgtatatgcacacctactgtcaagaggggcctggtcaatctattacacctcgaattttggctaaactgttcccagaattataa

SEQ ID NO: 12 - protein, 750 aa, Streptococcus agalactiae
MIIDRLLQRSHSHLPILQATFGLERESLRIHQPTQRVAQTPHPKTLGSRNYHPYIQTDYSEPQLELITPIAKDSQEAIRFLKAISDVAGRSINHDEYLWPLSMPPKVREEDIQIAQLEDAFEYDYRKYLEKTYGKLIQSISGIHYNLGLGQELLTSLFELSQADNAIDFQNQLYMKLSQNFLRYRWLLTYLYGASPVAEEDFLDQKLNNPVRSLRNSHLGYVNHKDIRISYTSLKDYVNDLENAVKSGQLIAEKEFYSPVRLRGSKACRNYLEKGITYLEFRTFDLNPFSPIGITQETVDTVHLFLLALLWIDSSSHIDQDIKEANRLNDLIALSHPLEKLPNQAPVSDLVDAMQSVIQHFNLSPYYQDLLESVKRQIQSPELTVAGQLLEMIEGLSLETFGQRQGQIYHDYAWEAPYALKGYETMELSTQLLLFDVIQKGVNFEVLDEQDQFLKLWHNSHIEYVKNGNMTSKDNYIVPLAMANKVVTKKILDEKHFPTPFGDEFTDRKEALNYFSQIQDKPIVVKPKSTNFGLGISIFKTSANLASYEKAIDIAFTEDSAILVEEYIEGTEYRFFVLEGDCIAVLLRVAANVVGDGIHTISQLVKLKNQNPLRGYDHRSPLEVIELGEVEQLMLEQQGYTVNSIPPEGTKIELRRNSNISTGGDSIDVTNTMDPTYKQLAAEMAEAMGAWVCGVDLIIPNATQAYSKDKKNATCIELNFNPLMYMHTYCQEGPGQSITPRILAKLFPEL

SEQ ID NO: 13 - DNA, 645 bp, Escherichia coli
atgcgtatcattctgcttggcgctccgggcgcggggaaagggactcaggctcagttcatcatggagaaatatggtattccgcaaatctccactggcgatatgctgcgtgctgcggtcaaatctggctccgagctgggtaaacaagcaaaagacattatggatgctggcaaactggtcaccgacgaactggtgatcgcgctggttaaagagcgcattgctcaggaagactgccgtaatggtttcctgttggacggcttcccgcgtaccattccgcaggcagacgcgatgaaagaagcgggcatcaatgttgattacgttctggaattcgacgtaccggacgaactgatcgttgaccgtatcgtcggtcgccgcgttcatgcgccgtctggtcgtgtttatcacgttaaattcaatccgccgaaagtagaaggcaaagacgacgttaccggtgaagaactgactacccgtaaagatgatcaggaagagaccgtacgtaaacgtctggttgaataccatcagatgacagcaccgctgatcggctactactccaaagaagcagaagcgggtaataccaaatacgcgaaagttgacggcaccaagccggttgctgaagttcgcgctgatctggaaaaaatcctcggctaa

SEQ ID NO: 14 - protein, 214 aa, Escherichia coli
MRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILG

SEQ ID NO: 15 - DNA, 1428 bp, Acinetobacter johnsonii
atggatacagaaacgatcgccagtgcagtgctgaatgaagaacagctttcactggacttaattgaagcgcaatatgcgttgatgaatacccgtgatcagagcaatgcaaaaagtttagtgattttggtcagtggaatcgaacttgcgggtaaaggcgaagcggtgaaacagctccgcgaatgggtcgatcctcgttttttatatgtcaaagccgatccaccgcatctgtttaatctaaaacagcctttttggcagccctatacccgatttgtgcctgccgaagggcaaattatggtgtggtttggtaattggtatggggatttgttggctacggccatgcatgcttcaaagcctttagatgacactttgtttgatgaatacgtcagcaatatgcgggcttttgaacaggacttaaaaaataacaacgtagatgtcttaaaagtttggttcgatttgtcgtggaagtctctgcaaaagcgtctagatgatatggacccgagcgaagtgcattggcataagttgcatgggctagactggcgcaataaaaaacaatatgacaccttacaaaagctacgtacgcgcttcaccgatgactggcaaatcattgatggtgaagatgaggatttgcgtaatcacaattttgcacaagcaattttaacggcactacgacactgcccagagcatgaaaaaaaggccgcgctaaaatggcagcaagcaccaataccagatattctgactcagtttgaagtccctcaagctgaggatgcgaactataaatcagaattgaaaaaactcaccaaacaagtggccgatgccatgcgctgtgatgaccgtaaagtggtgattgcttttgaaggtatggatgctgcgggtaaagggggggcgattaagcgtattgtgaaaaagctcgacccacgagaatatgaaattcataccattgccgcacctgaaaaatatgagttacgccgtccttatctgtggcgtttttggagcaaattgcagtcggatgacatcactatttttgatcggacgtggtatggacgcgttttagtcgagcgggtagaaggcttcgcaaccgaggtagagtggcaacgcgcttatgcggaaatcaatcgttttgaaaaaaacctcagtagcagccaaaccgtgctgattaagttttggctggcgattgataaagatgaacaagcagcgcgttttaaagcccgcgaaagtactccgcataaacgttttaaaattaccgaagaagattggcgcaatcgcgacaaatgggatgactatttaaaggcagccgcggatatgtttgcgcataccgacaccagctatgcgccttggtatattatttccaccaatgataaacaacaggcccgcattgaagtcttaagggcaattttaaaacagctcaaagcggatcgcgacacggattaa

SEQ ID NO: 16 - protein, 475 aa, Acinetobacter johnsonii
MDTETIASAVLNEEQLSLDLIEAQYALMNTRDQSNAKSLVILVSGIELAGKGEAVKQLREWVDPRFLYVKADPPHLFNLKQPFWQPYTRFVPAEGQIMVWFGNWYGDLLATAMHASKPLDDTLFDEYVSNMRAFEQDLKNNNVDVLKVWFDLSWKSLQKRLDDMDPSEVHWHKLHGLDWRNKKQYDTLQKLRTRFTDDWQIIDGEDEDLRNHNFAQAILTALRHCPEHEKKAALKWQQAPIPDILTQFEVPQAEDANYKSELKKLTKQVADAMRCDDRKVVIAFEGMDAAGKGGAIKRIVKKLDPREYEIHTIAAPEKYELRRPYLWRFWSKLQSDDITIFDRTWYGRVLVERVEGFATEVEWQRAYAEINRFEKNLSSSQTVLIKFWLAIDKDEQAARFKARESTPHKRFKITEEDWRNRDKWDDYLKAAADMFAHTDTSYAPWYIISTNDKQQARIEVLRAILKQLKADRDTD

(19) United States
(12) Patent Application Publication
(10) Pub. No.: US 2017/0211117 A1
(43) Pub. Date: Jul. 27, 2017

(54) METHOD FOR PRODUCING GAMMA-GLUTAMYLCYSTEINE AND GLUTATHIONE

(71) Applicant: KANEKA CORPORATION, Osaka-shi (JP)

(72) Inventors: Masutoshi NOJIRI, Hyogo (JP); Yoshihiko YASOHARA, Hyogo (JP)

(73) Assignee: KANEKA CORPORATION, Osaka-shi (JP)

(21) Appl. No.: 15/325,891

(22) PCT Filed: Jul. 28, 2015
(86) PCT No.: PCT/JP2015/071358

§ 371 Date: Jan. 12, 2017

(30) Foreign Application Priority Data
Jul. 29, 2014 (JP) 2014-154026

(51) Int. Cl. C12P 21/02; C12N 9/12; C12N 9/00

(52) U.S. Cl. CPC C12P 21/02; C12N 9/93; C12Y 603/02002; C12Y 603/02003; C12N 9/1229; C12Y 207/04

(57) ABSTRACT

The technical problem to be solved by the present invention is to provide a method for producing glutathione and a precursor thereof, γ-glutamylcysteine, at a high yield. As a means for solving the problem, the method for producing glutathione according to the present invention includes step A′ of reacting L-cysteine and L-glutamic acid under a low-oxygen atmosphere to produce γ-glutamylcysteine and step B′ of reacting γ-glutamylcysteine and glycine under a low-oxygen atmosphere to produce glutathione.

### TECHNICAL FIELD

The present invention relates to a method for producing γ-glutamylcysteine.

The present invention also relates to a method for producing glutathione.

### BACKGROUND ART

Glutathione is a peptide consisting of three amino acids, i.e., L-cysteine, L-glutamic acid and glycine and present in a wide variety of living organisms including not only humans but also other animals, plants and microorganisms, and is an important compound for living organisms involved in, e.g., removal of reactive oxygen species, detoxification and amino acid metabolism.

Glutathione is present in vivo in the form of either one of reduced glutathione (N-(N-γ-L-glutamyl-L-cysteinyl)glycine, in which the thiol group of the L-cysteine residue is reduced in the form of SH (hereinafter will be sometimes referred to as “GSH”) and oxidized glutathione in which the thiol group of the L-cysteine residue is oxidized to form a disulfide bond between two glutathione molecules (hereinafter will be sometimes referred to as “GSSG”).

Examples of a method for producing glutathione typically include, e.g., a fermentation method (Non Patent Literature 1) using saccharomyces cerevisiae and candida midis, and enzymatic methods (e.g., Patent Literatures 1, 2)(Non Patent Literatures 1, 2) using cells of Escherichia coli or saccharomyces cerevisiae, which are modified to produce γ-glutamylcysteine synthetase and glutathione synthetase by recombination technique, as an enzymatic source, in the presence of L-glutamic acid, L-cysteine, glycine, a surfactant and an organic solvent.

### CITATION LIST

### Patent Literatures

Patent Literature 1: JP Patent Publication (Kokai) No. 60-27396A (1985)

Patent Literature 2: JP Patent Publication (Kokai) No. 60-27397A (1985)

### Non Patent Literature

Non Patent Literature 1: Appl. Microbial. Biotechnol., 66, 233 (2004)

Non Patent Literature 2: Appl. Environ. Microbial., 44, 1444 (1982)

### SUMMARY OF INVENTION

### Technical Problem

The present inventors found a problem in the step of enzymatically producing γ-glutamylcysteine from L-cysteine and L-glutamic acid under the air atmosphere: not only γ-glutamylcysteine, but also oxidized γ-glutamylcysteine, which is a compound obtained by oxidizing the thiol groups of L-cysteine residues of two γ-glutamylcysteine molecules and binding them via a disulfide bond, is produced in a considerable amount, with the result that the yield of γ-glutamylcysteine decreases.

The present inventors found another problem in the step of enzymatically synthesizing glutathione from γ-glutamylcysteine and glycine under the air atmosphere: not only reduced glutathione but also a by-product, i.e., oxidized glutathione, is produced in a considerable amount, with the result that the yield of glutathione decreases.

The same problems may be present in the cases where the reaction is non-enzymatic.

A technical problem to be solved by the present invention is to provide a method for producing γ-glutamylcysteine while suppressing production of a by-product, i.e., oxidized γ-glutamylcysteine. Another technical problem to be solved by the present invention is to provide a method for producing glutathione while suppressing production of a by-product, i.e., oxidized glutathione.

In the present invention, “glutathione” or “GSH” exclusively means “reduced glutathione”; whereas, oxidized glutathione is referred to as “oxidized glutathione” or “GSSG”. In the present invention, “γ-glutamylcysteine” exclusively means “reduced γ-glutamylcysteine”; whereas, a compound obtained by binding two γ-glutamylcysteine molecules via an -S-S-bond through oxidation is referred to as “oxidized γ-glutamylcysteine”.

### Solution to Problem

In the specification, the following inventions are disclosed as the solutions to solve the aforementioned problems:

(1) A method for producing γ-glutamylcysteine, comprising step A of reacting L-cysteine and L-glutamic acid under an atmosphere having a lower oxygen concentration than atmospheric air, by the action of at least one enzyme selected from the group consisting of γ-glutamylcysteine synthetase and a bifunctional glutathione synthetase in the presence of adenosine triphosphate (ATP, also referred to as adenosine 5′-triphosphate), to produce γ-glutamylcysteine.

In the method (1), γ-glutamylcysteine can be produced with high efficiency while suppressing the production of a by-product, i.e., oxidized γ-glutamylcysteine.

(2) The method according to (1), wherein the step A is carried out in conjugation with an ATP regeneration reaction for regenerating adenosine diphosphate (ADP, also referred to as adenosine 5′-diphosphate) into adenosine triphosphate (ATP).

In the method (2), ATP consumed in the step A can be regenerated and the addition amount of ATP can be reduced.

(3) The method according to (1) or (2), wherein the γ-glutamylcysteine synthetase is derived from Escherichia coli.

The γ-glutamylcysteine synthetase used in the method (3) is preferable since it has a particularly high activity to produce γ-glutamylcysteine from L-cysteine and L-glutamic acid. [0019] (4) The method according to (1) or (2), wherein the bifunctional glutathione synthetase is derived from Streptococcus agalactiae.

The bifunctional glutathione synthetase used in the method (4) is preferable since it has a particularly high activity to produce γ-glutamylcysteine from L-cysteine and L-glutamic acid.

(5) A method for producing glutathione, comprising step B of reacting γ-glutamylcysteine and glycine under an atmosphere having a lower oxygen concentration than atmospheric air by the action of at least one enzyme selected from the group consisting of glutathione synthetase and a bifunctional glutathione synthetase in the presence of adenosine triphosphate (ATP), to produce glutathione.

In the method (5), production of a by-product, i.e., oxidized glutathione, can be suppressed and glutathione can be produced with high efficiency.

(6) The method according to (5), wherein the step B is carried out in conjugation with an ATP regeneration reaction for regenerating adenosine diphosphate (ADP) into adenosine triphosphate (ATP).

In the method (6), ATP consumed in the step B can be regenerated and the addition amount of ATP can be reduced.

(7) The method according to (5) or (6), wherein the glutathione synthetase is derived from Escherichia coli.

The glutathione synthetase used in the method (7) is preferable since it has a particularly high activity to produce glutathione from γ-glutamylcysteine and glycine.

(8) The method according to (5) or (6), wherein the bifunctional glutathione synthetase is derived from Streptococcus agalactiae.

The bifunctional glutathione synthetase used in the method (8) is preferable since it has a particularly high activity to produce glutathione from γ-glutamylcysteine and glycine.

(9) The method according to any one of (5) to (8), further comprising step A of reacting L-cysteine and L-glutamic acid under an atmosphere having a lower oxygen concentration than atmospheric air, by the action of at least one enzyme selected from the group consisting of γ-glutamylcysteine synthetase and a bifunctional glutathione synthetase in the presence of adenosine triphosphate (ATP), to produce γ-glutamylcysteine, wherein the γ-glutamylcysteine used in the step B is produced by the step A.

According to the method (9), the amounts of by-products, i.e., oxidized γ-glutamylcysteine and oxidized glutathione, are small and glutathione can be efficiently produced from L-cysteine and L-glutamic acid.

(10) The method according to (9), wherein the step A is carried out in conjugation with an ATP regeneration reaction for regenerating adenosine diphosphate (ADP) into adenosine triphosphate (ATP).

According to the method (10), ATP consumed in the step A can be regenerated and the addition amount of ATP can be reduced.

(11) The method according to (9) or (10), wherein the γ-glutamylcysteine synthetase is derived from Escherichia coli.

γ-glutamylcysteine synthetase used in the method (11) is preferable since it has a particularly high activity to produce γ-glutamylcysteine from L-cysteine and L-glutamic acid.

(12) The method according to (9) or (10), wherein the bifunctional glutathione synthetase is derived from Streptococcus agalactiae.

The bifunctional glutathione synthetase used in the method (12) is preferable since it has a particularly high activity to produce γ-glutamylcysteine from L-cysteine and L-glutamic acid.

(13) A method for producing γ-glutamylcysteine, comprising step A′ of reacting L-cysteine and L-glutamic acid under an atmosphere having a lower oxygen concentration than atmospheric air to produce γ-glutamylcysteine.

In the method (13), production of a by-product, i.e., oxidized γ-glutamylcysteine, can be suppressed and γ-glutamylcysteine can be produced with high efficiency. In this method, more preferably, the step A′ is the step A according to (1).

(14) A method for producing glutathione, comprising step B′ of reacting γ-glutamylcysteine and glycine under an atmosphere having a lower oxygen concentration than atmospheric air to produce glutathione.

In the method of (14), production of a by-product, i.e., oxidized glutathione, is suppressed and glutathione can be produced with high efficiency. In this method, more preferably, the step B′ is the step B according to (5).

(15) The method according to (14), further comprising step A′ of reacting L-cysteine and L-glutamic acid under an atmosphere having a lower oxygen concentration than atmospheric air to produce γ-glutamylcysteine, wherein the γ-glutamylcysteine used in the step B′ is produced by the step A′.

In the method (15), production of by-products, i.e., oxidized γ-glutamylcysteine and oxidized glutathione, is suppressed and glutathione can be produced with high efficiency. In this method, more preferably the step A′ is the step A according to (1).

In the specification, the compounds represented by terms: e.g., “L-cysteine”, “L-glutamic acid”, “glycine”, “γ-glutamylcysteine”, “glutathione”, “L-cystine”, “ oxidized γ-glutamylcysteine”, “oxidized glutathione”, “adenosine triphosphate”, “adenosine diphosphate”, “adenosine monophosphate”, “condensed phosphoric acid” and “polyphosphoric acid” may take any forms including free form, a salt (e.g., a sodium salt and a potassium salt), a solvate (e.g., a hydrate) and an ion.

In the present invention, “atmospheric air” refers to the atmospheric air on the earth and more specifically refers to “air”.

The specification includes the disclosure of Japanese Patent Application No. 2014-154026, on which the priority of the present application is based.

### Advantageous Effects of Invention

The present invention provides a method for producing γ-glutamylcysteine while suppressing by-production of oxidized γ-glutamylcysteine. The present invention further provides a method for producing glutathione while suppressing by-production of oxidized glutathione.

### DESCRIPTION OF EMBODIMENTS

### <Enzymes Used in the Present Invention>

As used herein, γ-glutamylcysteine synthetase will be sometimes referred to simply as “GSH I”, glutathione synthetase as “GSH II”, a bifunctional glutathione synthetase as “GSH F”, adenylate kinase as “ADK”, and polyphosphate-dependent AMP transferase as “PAP”.

### <GSH I>

The γ-glutamylcysteine synthetase (GSH I) used in the present invention is an enzyme having an activity to recognize L-cysteine (L-Cys) as a substrate and to catalyze a reaction for producing γ-Glu-Cys by mediating binding the L-cysteine (L-Cys) to L-glutamic acid (L-Glu) in the presence of ATP. GSH I may be any enzyme having this activity. The source, structure and other characteristics of GSH I are not limited. In the present invention, this activity is referred to as γ-glutamylcysteine synthetase activity. One unit (1 U) of the activity means the activity to produce 1 μmol of γ-glutamylcysteine at 30° C. for one minute and is measured in the following measurement condition.

### (Measurement Condition)

The reaction comprises adding an enzyme solution to a 50 mM tris hydrochloride buffer solution (pH8.0) containing 10 mM ATP, 15 mM L-glutamic acid, 15 mM L-cysteine and 10 mM magnesium sulfate and then keeping the reaction solution at 30° C. The reaction is terminated by adding 6N hydrochloric acid. The γ-glutamylcysteine in the reaction solution is quantified by high-performance liquid chromatography.

The conditions for the high-performance liquid chromatography are as follows. In the conditions, glutathione (GSH), γ-glutamylcysteine (γ-GC), oxidized γ-GC and oxidized glutathione (GSSG) sequentially elute in this order.

### [HPLC Conditions]

Column: ODS-HG-3 (4.6 mmφ×150 mm, manufactured by Nomura Chemical Co., Ltd.);

Eluent: Solution prepared by dissolving potassium dihydrogenphosphate (12.2 g) and sodium heptanesulfonate (3.6 g) with distilled water (1.8 L), controlling the pH of the solution with phosphoric acid to be pH2.8, adding methanol (186 ml) and dissolving it;

Flow rate: 1.0 ml/minute;

Column temperature: 40° C.;

Measurement wavelength: 210 nm.

GSH I having a γ-glutamylcysteine synthetase activity (specific activity) of 0.5 U or more per 1 mg of protein is preferably used as GSH I.

The source from which GSH I is derived is not limited and GSH I derived from e.g., microorganisms, animals and plants can be used. GSH I derived from a microorganism is preferable, particularly GSH I derived from an intestinal microbe, such as Escherichia coli, a corynebacterium and an eukaryotic microorganism, such as a yeast, are preferable.

The nucleotide sequence of GSH I derived from Escherichia coli and the amino acid sequence encoded by the nucleotide sequence are specifically represented by SEQ ID NO: 1 and SEQ ID NO: 9, respectively.

GSH I that can be used includes, not only GSH I consisting of the amino acid sequence represented by SEQ ID NO: 9 but also other polypeptides having GSH I activity, such as an active mutant thereof and orthologue from other species. Said other polypeptides having GSH I activity includes a polypeptide exhibiting an activity of preferably 10% or more, preferably 40% or more, more preferably 60% or more, more preferably 80% or more and further preferably 90% or more relative to the activity of the GSH I consisting of the amino acid sequence represented by SEQ ID NO: 9 in the aforementioned activity measurement conditions. Examples of said other polypeptides having GSH I activity, such as an active mutant thereof and orthologue from other species, include a polypeptide consisting of an amino acid sequence having addition, deletion or substitution of one or several amino acids in the amino acid sequence represented by SEQ ID NO: 9 (particularly preferably, a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 9); and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 9. Further examples of said other polypeptides that can be used herein include a GSH I-active fragment of at least one polypeptide selected from the group consisting of the polypeptide consisting of the amino acid sequence represented by SEQ ID NO: 9; a polypeptide consisting of an amino acid sequence having addition, deletion or substitution of one or several amino acids in the amino acid sequence represented by SEQ ID NO: 9; and a polypeptide consisting of the amino acid sequence having the aforementioned amino acid identity with the amino acid sequence represented by SEQ ID NO: 9. The fragment may be a polypeptide consisting of amino acids of preferably 250 or more, more preferably 300 or more, more preferably 400 or more and more preferably 500 or more. In the specification, “several” refers to, for example, 2 to 20, 2 to 15, 2 to 10, 2 to 7, 2 to 5, 2 to 4 or 2 to 3. The “amino acid identity” refers to the percentage (%) of the number of identical amino acid residues relative to the total number of amino acid residues of the protein represented by SEQ ID NO: 9, when two amino acid sequences are aligned so as to obtain a highest degree of matching of amino acids between the two sequences, if necessary by introducing a gap. The amino acid identity can be calculated by using a protein search system, such as BLAST and FASTA (Karlin, S. et al., 1993, Proc. Natl. Acad. Sci. USA, 90: 5873-5877; Altschul, S. F. et al., 1990, J. Mol. Biol., 215: 403-410; Pearson, W.R. et al., 1988, Proc. Natl. Acad. Sci. USA, 85: 2444-2448). Furthermore, the substitution of amino acids is made preferably in accordance with conservative amino acid substitution. The “conservative amino acid substitution” refers to substitution between amino acids having analogous properties with respect to e.g., charge, side chain, polarity and aromaticity. The amino acids analogous in property are categorized as follows: a basic amino acid (arginine, lysine, histidine); an acidic amino acid (aspartic acid, glutamic acid); an uncharged polar amino acid (glycine, asparagine, glutamine, serine, threonine, cysteine, tyrosine); a nonpolar amino acid (leucine, isoleucine, alanine, valine, proline, phenylalanine, tryptophan, methionine); a branched-chain amino acid (leucine, valine, isoleucine); and an aromatic amino acid (phenylalanine, tyrosine, tryptophan, histidine). The aforementioned polypeptides each may be chemically modified.

The nucleotide sequence of a gene (DNA or RNA) encoding GSH I that can be used for preparation of GSH I is not limited to the nucleotide sequence represented by SEQ ID NO: 1. The nucleotide sequence may be any nucleotide sequence encoding the amino acid sequence of a desired GSH I and being suited to the host organism.

### <GSH II>

The glutathione synthetase (GSH II) used in the present invention is an enzyme having an activity to recognize γ-Glu-Cys as a substrate and to catalyze a reaction for producing γ-Glu-Cys-Gly by mediating binding the γ-Glu-Cys to glycine (Gly) in the presence of ATP. GSH II may be any enzyme having this activity. The source, structure and other characteristics of GSH II are not limited. In the present invention, the activity is referred to as glutathione synthetase activity. One unit (1 U) of the activity means the activity to produce 1 μmol of glutathione at 30° C. for one minute and is measured in the following measurement conditions.

### (Measurement Condition)

The reaction comprises adding an enzyme solution to a 50 mM tris hydrochloride buffer solution (pH8.0) containing 10 mM ATP, 15 mM γ-glutamylcysteine, 15 mM glycine and 10 mM magnesium sulfate and then keeping the reaction solution at 30° C. The reaction is terminated by adding 6N hydrochloric acid. The glutathione in the reaction solution is quantified by high-performance liquid chromatography.

The same conditions for the high-performance liquid chromatography as mentioned in the above GSH I activity measurement method are used.

GSH II having a glutathione synthetase activity (specific activity) of 0.5 U or more per 1 mg of protein is preferably used.

The source from which GSH II is derived is not limited and GSH II derived from e.g., microorganisms, animals and plants can be used. GSH II derived from a microorganism is preferable, particularly GSH II derived from an intestinal microbe, such as Escherichia coli, a corynebacterium and an eukaryotic microorganism, such as yeasts, are preferable.

The nucleotide sequence of GSH II derived from Escherichia coli and the amino acid sequence encoded by the nucleotide sequence are specifically represented by SEQ ID NO: 4 and SEQ ID NO: 10, respectively.

GSH II that can be used includes not only GSH II consisting of the amino acid sequence represented by SEQ ID NO: 10 but also other polypeptides having GSH II activity, such as an active mutant thereof and orthologue from other species. Said other polypeptides having GSH II activity include a polypeptide exhibiting an activity of preferably 10% or more, preferably 40% or more, more preferably 60% or more, more preferably 80% or more and further preferably 90% or more relative to the activity of the GSH II consisting of the amino acid sequence represented by SEQ ID NO: 10 in the aforementioned activity measurement conditions. Examples of said other polypeptides having GSH II activity, such as an active mutant thereof and orthologue from other species, include a polypeptide consisting of an amino acid sequence having addition, deletion or substitution of one or several amino acids in the amino acid sequence represented by SEQ ID NO: 10 (particularly preferably, a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 10); and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 10. Further examples of said other polypeptides that can be used herein include a GSH II-active fragment of at least one polypeptide selected from the group consisting of the polypeptide consisting of the amino acid sequence represented by SEQ ID NO: 10; a polypeptide consisting of an amino acid sequence having addition, deletion or substitution of one or several amino acids in the amino acid sequence represented by SEQ ID NO: 10; and a polypeptide consisting of the amino acid sequence having the aforementioned amino acid identity with the amino acid sequence represented by SEQ ID NO: 10. The fragment may be a polypeptide consisting of amino acids of preferably 150 or more, more preferably 200 or more, more preferably 300 or more. The “amino acid identity” refers to the percentage (%) of the number of identical amino acid residues relative to the total number of amino acid residues of the protein represented by SEQ ID NO: 10, when two amino acid sequences are aligned and arranged so as to obtain a highest degree of matching of amino acids between the two sequences, if necessary by introducing a gap. Furthermore, the substitution of amino acids is made preferably in accordance with conservative amino acid substitution. The preferable range of “several”, method for calculating amino acid identity and conservative amino acid substitution mentioned herein are the same as described with respect to GSH I. The aforementioned polypeptides each may be chemically modified.

The nucleotide sequence of a gene (DNA or RNA) encoding the GSH II that can be used for preparation of GSH II is not limited to the nucleotide sequence represented by SEQ ID NO: 4. The nucleotide sequence may be any nucleotide sequence encoding the amino acid sequence of a desired GSH II and being suited to the host organism.

### <GSH F>

The bifunctional glutathione synthetase (GSH F) used in the present invention is an enzyme having two activities in combination, one of which is an activity to recognize L-Cys as a substrate and to catalyze a reaction for producing γ-Glu-Cys by mediating binding the L-Cys to L-Glu in the presence of ATP, and the other of which is an activity to recognize γ-Glu-Cys as a substrate and to catalyze a reaction for producing γ-Glu-Cys-Gly by mediating binding the γ-Glu-Cys to Gly in the presence of ATP. GSH F may be any enzyme having these two activities in combination. The source, structure and other characteristics of GSH F are not limited. In the present invention, the combination of the activities is referred to as bifunctional glutathione synthetase activity. One unit (1 U) of the activity means the activity to produce 1 μmol of γ-Glu-Cys-Gly (glutathione) at 30° C. for one minute and is measured in the following measurement conditions.

### (Measurement Condition)

The reaction comprises adding an enzyme solution to a 50 mM tris hydrochloride buffer solution (pH8.0) containing 10 mM ATP, 15 mM L-glutamic acid, 15 mM L-cysteine, 15 mM glycine and 10 mM magnesium sulfate and then keeping the reaction solution at 30° C. The reaction is terminated by adding 6N hydrochloric acid. The glutathione in the reaction solution is quantified by high-performance liquid chromatography.

The same conditions for the high-performance liquid chromatography as mentioned in GSH I activity measurement method are used.

GSH F having a bifunctional glutathione synthetase activity (specific activity) of 0.5 U or more per 1 mg of protein is preferably used.

The source from which GSH F is derived is not limited and GSH F derived from e.g., microorganisms, animals and plants can be used. GSH F derived from a microorganism is preferable, particularly GSH F derived from a bacterium, and more specifically GSH F derived from at least one selected from the group consisting of Streptococcus bacteria, such as Streptococcus agalactiae, Streptococcus mulans, Streptococcus suis and Streptococcus thermophiles, Lactobacillus bacteria, such as Lactobacillus plantarum; Desulfotalea bacteria, such as Desulfotalea psychrophila; Clostridium bacteria, such as Clostridium perfringens; Listeria bacteria, such as Listeria innocua and Listeria monocytogenes; Enterococcus bacteria, such as Enterococcus faecalis and Enterococcus faecium; Pasteurella bacteria, such as Pasteurella multocida; Mannheimia bacteria, such as Mannheimia succiniciprodecens; and Haemophilus bacteria, such as Haemophilus somnus, is preferable.

The nucleotide sequence of GSH F derived from Streptococcus agalactiae and the amino acid sequence encoded by the nucleotide sequence are specifically represented by SEQ ID NO: 11 and SEQ ID NO: 12, respectively. Furthermore, the nucleotide sequence consisting of 4th base to 2253rd base of SEQ ID NO: 7 is an example of a nucleotide sequence encoding GSH F derived from Streptococcus agalactiae consisting of the amino acid sequence represented by SEQ ID NO: 12 and being suited in accordance with the frequency of codons in Escherichia coli.

GSH F that can be used includes not only GSH F consisting of the amino acid sequence represented by SEQ ID NO: 12 but also other polypeptides having GSH F activity, such as an active mutant thereof and orthologue from other species. Said other polypeptides having GSH F activity includes a polypeptide exhibiting an activity of preferably 10% or more, preferably 40% or more, more preferably 60% or more, more preferably 80% or more and further preferably 90% or more relative to the activity of the GSH F consisting of the amino acid sequence represented by SEQ ID NO: 12 in the aforementioned activity measurement conditions. Examples of said other polypeptides having GSH F activity, such as an active mutant thereof and orthologue from other species, include a polypeptide consisting of an amino acid sequence having addition, deletion or substitution of one or several amino acids in the amino acid sequence represented by SEQ ID NO: 12 (particularly preferably, a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 12); and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 12. Further examples of said other polypeptides that can be used herein include a GSH F-active fragment of at least one polypeptide selected from the group consisting of the polypeptide consisting of the amino acid sequence represented by SEQ ID NO: 12; a polypeptide consisting of an amino acid sequence having addition, deletion or substitution of one or several amino acids in the amino acid sequence represented by SEQ ID NO: 12; and a polypeptide consisting of the amino acid sequence having the aforementioned amino acid identity with the amino acid sequence represented by SEQ ID NO: 12. The fragment may be a polypeptide consisting of amino acids of preferably 400 or more, more preferably 500 or more, and more preferably 600 or more, more preferably 700 or more and more preferably 730 or more. The “amino acid identity” refers to the percentage (%) of the number of identical amino acid residues relative to the total number of amino acid residues of the protein represented by SEQ ID NO: 12 when two amino acid sequences are aligned and arranged so as to obtain a highest degree of matching of amino acids between the two sequences, if necessary by introducing a gap. Furthermore, the substitution of amino acids is made preferably in accordance with conservative amino acid substitution. The preferable range of “several”, method for calculating amino acid identity and conservative amino acid substitution mentioned herein are the same as described with respect to GSH I. The aforementioned polypeptides each may be chemically modified.

The nucleotide sequence of a gene (DNA or RNA) encoding the GSH F that can be used for preparation of GSH F is not limited to the nucleotide sequence represented by SEQ ID NO: 11. The nucleotide sequence may be any nucleotide sequence encoding the amino acid sequence of a desired GSH F and being suited to the host organism.

### <ADK>

Adenylate kinase (ADK) used in the present invention is an enzyme having an activity to catalyze a reaction for producing one ATP molecule and one AMP molecule from two ADP molecules. ADK may be any enzyme having this activity. The source, structure and other characteristics of ADK are not limited. In the present invention, the activity is referred to as ADK activity. One unit (1 U) of the activity means the activity to produce 1 μmol of AMP at 30° C. for one minute and is measured in the following measurement conditions.

### (Measurement Condition)

The reaction comprises adding an enzyme solution to a 50 mM tris hydrochloride buffer solution (pH8.0) containing 10 mM ADP and 70 mM magnesium sulfate, and then keeping the reaction solution at 30° C. The reaction is terminated by adding 6N hydrochloric acid. The AMP in the reaction solution is quantified by high-performance liquid chromatography.

The conditions of the high-performance liquid chromatography are as follows. In the conditions, adenosine triphosphate (ATP), adenosine diphosphate (ADP) and adenosine monophosphate (5′-adenyl acid) (AMP) sequentially elute in this order.

### [HPLC Conditions]

Column: ODS-HG-3 (4.6 mmφ×150 mm, manufactured by Nomura Chemical Co., Ltd.);

Eluent: Solution prepared by dissolving potassium dihydrogenphosphate (12.2 g) and sodium heptanesulfonate (3.6 g) with distilled water (1.8 L), controlling the pH of the solution with phosphoric acid to be pH2.8, adding methanol (186 ml) and dissolving it;

Flow rate: 1.0 ml/minute;

Column temperature: 40° C.;

Measurement wavelength: 210 nm.

ADK having an ADK activity (specific activity) of 20 U or more per 1 mg of protein is preferably used.

The source from which ADK is derived is not limited and ADK derived from e.g., microorganisms, animals and plants can be used. ADK derived from a microorganism is preferable, and particularly ADK derived from a bacterium, and more specifically ADK derived from Escherichia coli is preferable.

The nucleotide sequence of ADK derived from Escherichia coli and the amino acid sequence encoded by the nucleotide sequence are specifically represented by SEQ ID NO: 13 and SEQ ID NO: 14, respectively.

ADK that can be used includes not only ADK consisting of the amino acid sequence represented by SEQ ID NO: 14 but also other polypeptides having ADK activity, such as an active mutant thereof and orthologue from other species. Said other polypeptides having ADK activity is preferably a polypeptide exhibiting an activity of preferably 10% or more, preferably 40% or more, more preferably 60% or more, more preferably 80% or more and further preferably 90% or more relative to the activity of the ADK consisting of the amino acid sequence represented by SEQ ID NO: 14 in the aforementioned activity measurement conditions. Examples of said other polypeptides having ADK activity, such as an active mutant thereof and orthologue from other species, include a polypeptide consisting of an amino acid sequence having addition, deletion or substitution of one or several amino acids in the amino acid sequence represented by SEQ ID NO: 14 (a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 14) and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 14. Further examples of said other polypeptides that can be used herein include a ADK-active fragment of at least one polypeptide selected from the group consisting of the polypeptide consisting of the amino acid sequence represented by SEQ ID NO: 14; a polypeptide consisting of an amino acid sequence having addition, deletion or substitution of one or several amino acids in the amino acid sequence represented by SEQ ID NO: 14; and a polypeptide consisting of the amino acid sequence having the aforementioned amino acid identity with the amino acid sequence represented by SEQ ID NO: 14. The fragment may be a polypeptide consisting of amino acids of preferably 100 or more, more preferably 150 or more, more preferably 200 or more. The “amino acid identity” refers to the percentage (%) of the number of identical amino acid residues relative to the total number of amino acid residues of the protein represented by SEQ ID NO: 14, when two amino acid sequences are aligned and arranged so as to obtain a highest degree of matching of amino acids between the two sequences, if necessary by introducing a gap. Furthermore, the substitution of amino acids is made preferably in accordance with conservative amino acid substitution. The preferable range of “several”, method for calculating amino acid identity and conservative amino acid substitution mentioned herein are the same as described with respect to GSH 1. The aforementioned polypeptides each may be chemically modified.

The nucleotide sequence of a gene (DNA or RNA) encoding the ADK that can be used for preparation of ADK is not limited to the nucleotide sequence represented by SEQ ID NO: 13. The nucleotide sequence may be any nucleotide sequence encoding the amino acid sequence of a desired ADK and being suited to the host organism.

### <PAP>

Polyphosphate-dependent AMP transferase (PAP) used in the present invention is an enzyme having an activity to catalyze a reaction for phosphorylating AMP using a polyphosphoric acid as a phosphoric acid donor to produce ADP. PAP may be any enzyme having this activity. The source, structure and other characteristics of PAP are not limited. In the present invention, the activity is referred to as PAP activity. One unit (1 U) of the activity means the activity to produce 1 μmol of ADP at 30° C. for one minute and is measured in the following measurement condition.

### (Measurement Condition)

The reaction comprises adding an enzyme solution to a 50 mM tris hydrochloride buffer solution (pH8.0) containing 5 mM sodium metaphosphate, 10 mM AMP and 70 mM magnesium sulfate and then keeping the reaction solution at 30° C. The reaction is terminated by adding 6N hydrochloric acid. ADP in the reaction solution is quantified by high-performance liquid chromatography.

The same conditions for the high-performance liquid chromatography as mentioned in the above activity measurement method for ADK are used.

PAP having a PAP activity (specific activity) of 20 U or more per 1 mg of protein is preferably used.

The source of PAP is not limited and PAP derived from e.g., microorganisms, animals and plants can be used. PAP derived from a microorganism is preferable, and particularly PAP derived from a bacterium, and more specifically PAP derived from Acinetobacter johnsonii is preferable.

The nucleotide sequence of PAP derived from Acinetobacter johnsonii and the amino acid sequence encoded by the nucleotide sequence are specifically represented by SEQ ID NO: 15 and SEQ ID NO: 16, respectively. Furthermore, the nucleotide sequence consisting of 4th base to 1428th base of SEQ ID NO: 8 is an example of a nucleotide sequence encoding PAP derived from Acinetobacter johnsonii consisting of the amino acid sequence represented by SEQ ID NO: 16, and being suited in accordance with the use frequency of codons in Escherichia coli.

PAP that can be used includes not only PAP consisting of the amino acid sequence represented by SEQ ID NO: 16 but also other polypeptides having PAP activity, such as an active mutant thereof and orthologue from other species. Said other polypeptides having PAP activity include a polypeptide exhibiting an activity of preferably 10% or more, preferably 40% or more, more preferably 60% or more, more preferably 80% or more and further preferably 90% or more relative to the activity of the PAP consisting of the amino acid sequence represented by SEQ ID NO: 16 in the aforementioned activity measurement conditions. Examples of said other polypeptides having PAP activity, such as an active mutant thereof and orthologue from other species, include a polypeptide consisting of an amino acid sequence having addition, deletion or substitution of one or several amino acids in the amino acid sequence represented by SEQ ID NO: 16 (particularly preferably, a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 16); and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 16. Further examples of said other polypeptides that can be used herein include a PAP-active fragment of at least one polypeptide selected from the group consisting of the polypeptide consisting of the amino acid sequence represented by SEQ ID NO: 16; a polypeptide consisting of an amino acid sequence having addition, deletion or substitution of one or several amino acids in the amino acid sequence represented by SEQ ID NO: 16; and a polypeptide consisting of the amino acid sequence having the aforementioned amino acid identity with the amino acid sequence represented by SEQ ID NO: 16. The fragment may be a polypeptide consisting of amino acids of preferably 250 or more, more preferably 300 or more, more preferably 400 or more and more preferably 450 or more. The “amino acid identity” refers to the percentage (%) of the number of identical amino acid residues relative to the total number of amino acid residues of the protein represented by SEQ ID NO: 16 when two amino acid sequences are aligned and arranged so as to obtain a highest degree of matching of amino acids between the two sequences, if necessary by introducing a gap. Furthermore, the substitution of amino acids is made preferably in accordance with conservative amino acid substitution. The preferable range of “several”, method for calculating amino acid identity and conservative amino acid substitution mentioned herein are the same as described with respect to GSH I. The aforementioned polypeptides each may be chemically modified.

The nucleotide sequence of a gene (DNA or RNA) encoding the PAP that can be used for preparation of PAP is not limited to the nucleotide sequence represented by SEQ ID NO: 15. The nucleotide sequence may be any nucleotide sequence encoding the amino acid sequence of a desired PAP and being suited to the host organism.

### <Preparation of Enzymes>

The methods for obtaining the aforementioned individual enzymes used in the present invention are not limited. Each of the enzymes can be prepared from organisms having the activity of the enzyme, for example, a wild strain or variant strain of a microorganism. As the organism having the activity of a desired enzyme, either an organism that natively has the enzymatic activity or an organism that has been augmented in the enzymatic activity may be used. Examples of the organism that has been augmented in the enzymatic activity include recombinant biological cells, in which the expression of a gene encoding each of the enzymes has been augmented by genetic engineering technique. Note that “the organism that has been augmented in the enzymatic activity” include not only an organism that natively has the enzymatic activity, in which the enzymatic activity has been augmented further, but also an organism that natively lacks in the enzymatic activity, in which the enzymatic activity has been added.

The recombinant biological cell obtained by genetic engineering technique typically refers to a recombinant biological cell having an ability to produce a desired enzyme. Such a cell can be obtained by inserting a gene (DNA or RNA) encoding the desired enzyme into an appropriate vector to prepare a recombinant vector and transforming an appropriate host cell with the recombinant vector. The desired enzyme can be produced by culturing the recombinant biological cell. Examples of the host cell include bacterial cells, yeast cells, filamentous fungus cells, plant cells and animal cells. In view of introduction and expression efficiency, bacterial cells are preferable and particularly Escherichia coli cell is preferable.

<Step A and step A′>

The method for producing β-glutamylcysteine according to the present invention is characterized by comprising step A of reacting L-cysteine and L-glutamic acid under an atmosphere having a lower oxygen concentration than atmospheric air, by the action of at least one enzyme selected from the group consisting of γ-glutamylcysteine synthetase (GSH 1) and a bifunctional glutathione synthetase (GSH F) in the presence of adenosine triphosphate (ATP), to produce γ-glutamylcysteine. Step A is a step of reacting L-cysteine and L-glutamic acid in the presence of the enzyme and ATP to produce γ-glutamylcysteine. When the enzyme acts and catalyzes the above reaction, adenosine triphosphate (ATP) is consumed.

The method for producing γ-glutamylcysteine according to the present invention is also characterized by comprising step A′ of reacting L-cysteine and L-glutamic acid under an atmosphere having a lower oxygen concentration than atmospheric air to produce γ-glutamylcysteine. Step A′ may be a step based on an enzymatic reaction or a step based on a chemical reaction using no enzyme; preferably a step based on an enzymatic reaction and particularly preferably the step A as mentioned above. The enzymatic reaction is advantageous over the chemical synthesis reaction, for the reason that e.g., protection of a substrate compound with a functional group is not required or specificity of a reaction is high.

Examples of step A′ based on a chemical reaction using no enzyme include, but not particularly limited to, a step comprising reacting L-cysteine having carboxyl groups protected with an appropriate protecting group with L-glutamic acid having an a-carboxyl group and an amino group protected with appropriate protecting groups and subjecting an amino group of one L-cysteine molecule and a γ-carboxyl group of one L-glutamic acid molecule to dehydration condensation to form a peptide bond. In the step, if necessary, one or more protecting groups are removed after the dehydration condensation reaction. The protecting group for a carboxyl group may be a commonly known protecting group for a carboxyl group, such as a benzyl group. The protecting group for an amino group may be a commonly known protecting group for an amino group, such as t-butoxycarbonyl (Boc) group and a 9-fluorenylmethoxycarbonyl (Fmoc) group.

Step A may use, as GSH I and/or GSH F, a cell of an organism having GSH I and/or GSH F activity, which may be a living cell or may be a dead but undamaged cell. Alternatively, the step may use GSH I and/or GSH F present outside the cell, more specifically, a ground material of the cell of the organism. Alternatively, the step may use GSH I and/or GSH F protein, which is isolated from the cell and appropriately purified as needed. Herein, the purification degree of a protein having GSH I and/or GSH F activity is not limited, and the step may use GSH I and/or GSH F roughly purified.

As the enzyme, step A preferably uses no living cell having GSH I and/or GSH F activity, and more preferably, uses neither a living cell nor an undamaged dead cell having GSH I and/or GSH F activity. In particular, step A preferably uses, as the enzyme, the extracellular GSH I and/or GSH F, more specifically, GSH I and/or GSH F present in a ground material of the cell or GSH I and/or GSH F protein isolated from the cell. In the case where step A uses a living cell having GSH I and/or GSH F activity, adenosine monophosphate (AMP) is easily decomposed in the reaction system (see, Example 4). Since AMP is one of the intermediates in the ATP regeneration reaction (described later), if AMP is decomposed, it is difficult to efficiently conduct the ATP regeneration reaction. In contrast, step A preferably uses extracellular GSH I and/or GSH F, since AMP is rarely decomposed and the ATP regeneration reaction can efficiently proceed. In the case where the reaction of step A is carried out without using a living cell, the reduction action by the living cell is absent in the reaction system. Without such reduction action, oxidation could easily proceed under an oxygen-rich atmosphere and oxidized γ-glutamylcysteine could be produced. However, in the present invention, the reaction of step A is carried out under an atmosphere having a lower oxygen concentration than atmospheric air. Owing to this, oxidation of γ-glutamylcysteine is suppressed and reduced γ-glutamylcysteine can be produced at a high yield. In an embodiment in which step A of the present invention uses extracellular GSH I and/or GSH F, more specifically, a ground material of a cell or isolated protein (from the cell) having GSH I and/or GSH F activity, decomposition of AMP and oxidation of γ-glutamylcysteine can be simultaneously suppressed.

In the specification, “grinding” of a cell refers to a treatment by which the surface structure of a cell is damaged to the extent that an enzyme produced within the cell is accessible from outside the cell, and the cell is not necessarily fragmented. In the specification, the “ground material” of a cell refers to a ground cell treated by grinding. Grinding of a cell can be carried out by applying one grinding treatment or a plurality of grinding treatments in an appropriate order. Examples of the grinding treatment of a cell include a physical treatment, a chemical treatment and an enzymatic treatment. Examples of the physical treatment include treatments using a high-pressure homogenizer, an ultrasonic homogenizer, a French press, a ball mill and a combination of these treatments. Examples of the chemical treatment include a treatment using an acid (preferably a strong acid), such as hydrochloric acid and sulfuric acid, a treatment using a base (preferably a strong base), such as sodium hydroxide and potassium hydroxide, and a combination of these treatments. Examples of the enzymatic treatment include treatments using, lysozyme, zymolyase, glucanase, protease and cellulase, and a combination of these treatments.

L-cysteine, L-glutamic acid and adenosine triphosphate (ATP) in step A and L-cysteine and L-glutamic acid in step A′ each can be added in the reaction system in any form, such as a salt, free form and a solvate (e.g., a hydrate).

It is preferable that L-cysteine to be used as a starting material in step A and/or step A′ does not substantially contain L-cystine. More specifically, L-cysteine is contained in a percentage of 70 mol % or more relative to the total molar amount of L-cystine and L-cysteine, more preferably 80 mol % or more, more preferably 90 mol % or more, further preferably 95 mol % or more, further preferably 98 mol % or more and most preferably 100 mol %. The requirement that the percentage of L-cysteine relative to the total molar amount of L-cysteine and L-cystine falls within the above range is satisfied preferably at least at the starting time of the reaction in step A and/or step A′ and more preferably during the period from the starting time to completion of the reaction in step A and/or step A′.

In step A and/or step A′, it is characterized in that reaction is carried out under “an atmosphere having a lower oxygen concentration than atmospheric air”. As the atmosphere, an atmosphere having an oxygen concentration of 10 vol % or less is preferable and an atmosphere having an oxygen concentration of 5 vol % or less is further preferable. The lower limit is not particularly limited and the oxygen concentration may be 0 vol %. The “atmosphere where an oxygen concentration is lower than atmospheric air” is, for example, an inert gas atmosphere. The inert gas is not limited as long as oxygen is not contained therein; however, an atmosphere of an inert gas, such as nitrogen, a noble gas (e.g., argon) and carbon dioxide gas, is preferable. The phrase “oxygen is not contained therein” in the specification also includes the case where substantially no oxygen is contained. A reaction in the atmosphere satisfying the above oxygen concentration can be realized by carrying out a reaction in a reaction vessel in which the gaseous phase is replaced with the inert gas as mentioned above. The phrase “carrying out a reaction in a reaction vessel in which the gaseous phase is replaced with the inert gas as mentioned above” also includes carrying out a reaction while supplying the inert gas flow as needed. The pressure of the atmosphere is not limited and the atmospheric pressure is usually about normal pressure and can be typically 0.08 to 0.12 MPa. If γ-glutamylcysteine is produced under such an atmosphere, production of a by-product, i.e., oxidized γ-glutamylcysteine, is suppressed and γ-glutamylcysteine can be efficiently produced. In step A and/or step A′, the yield of γ-glutamylcysteine relative to a substrate, L-cysteine, is typically 80 mol % or more, preferably 85 mol % or more, more preferably 90 mol % or more and particularly preferably 95 mol % or more. After completion of the reaction in step A and/or step A′, oxidized γ-glutamylcysteine is not substantially contained in the reaction system (for example, the reaction mixture). More specifically, the content of γ-glutamylcysteine relative to the total molar amount of γ-glutamylcysteine and oxidized γ-glutamylcysteine in the reaction system after completion of the reaction in step A and/or step A′, is 80 mol % or more, more preferably 90 mol % or more, further preferably 95 mol % or more, further preferably 97 mol % or more and most preferably 100 mol %.

In step A and/or step A′, the reaction can be carried out in a reaction mixture containing a solvent, such as water, controlled at appropriate pH. The conditions herein is not particularly limited; however, the concentration of a substrate (total concentration of L-cysteine and L-glutamic acid) can be preferably about 0.1 to 99 wt % and more preferably 1 to 20 wt %. The quantitative ratio of L-cysteine and L-glutamic acid in the substrate at the staring time of the reaction is about 1 mole of L-glutamic acid to 1 mole of L-cysteine, for example, the content of L-glutamic acid relative to 1 mole of L-cysteine can be 0.5 to 2 moles, and preferably 0.7 to 1.3 moles. The reaction temperature can be preferably 10 to 60° C. and more preferably 20 to 50° C. The pH of the reaction mixture is preferably 4 to 11 and more preferably 6 to 9. The reaction time can be preferably 1 to 120 hours and more preferably 1 to 72 hours.

The concentration of each of the enzymes in the reaction mixture can be appropriately controlled. For example, the lower limit thereof in terms of protein is 1 μg/ml or more and the upper limit is not specified; however, it can be appropriately controlled in the range up to preferably 100 mg/ml or less. When GSH I is used in step A, the GSH I activity in the reaction mixture in step A is not limited; however, the lower limit thereof is preferably 0.05 U/ml or more and the upper limit (not particularly specified) can be usually 5000 U/ml or less. When GSH F is used in step A, GSH F activity in the reaction mixture in step A is not limited; however, the lower limit thereof is preferably 0.05 U/ml or more and the upper limit (not particularly specified) can be set at usually 5000 U/ml or less.

The concentration of ATP in the reaction mixture can be appropriately controlled depending upon the concentration of L-cysteine serving as a substrate and the presence or absence of the ATP regenerating system. In step A, ATP is consumed in the molar amount equal to L-cysteine. In the case where step A is carried out in conjugation with ATP regeneration reaction, the addition amount of ATP relative to L-cysteine can be greatly reduced. Then, the upper limit of the ATP concentration in the reaction mixture in step A, which is not limited, is preferably 2 fold or less and more preferably 1.2 fold or less as large as the L-cysteine concentration by the molar concentration ratio. The lower limit of the ATP concentration in the reaction mixture in step A, which is not limited, is preferably 0.0001 fold or more, more preferably 0.001 fold or more and further preferably 0.01 fold or more as large as the L-cysteine concentration by the molar concentration ratio.

### <Step B and Step B′>

A method for producing glutathione (GSH) according to the present invention is characterized by comprising step B of reacting γ-glutamylcysteine and glycine under an atmosphere having a lower oxygen concentration than atmospheric air, by the action of at least one enzyme selected from the group consisting of glutathione synthetase (GSH II) and a bifunctional glutathione synthetase (GSH F) in the presence of adenosine triphosphate (ATP), to produce glutathione. Step B is a step of reacting γ-glutamylcysteine and glycine in the presence of the aforementioned enzyme and adenosine triphosphate (ATP) to produce glutathione, and ATP is consumed when the enzyme acts and catalyzes the above reaction.

The method for producing glutathione according to the present invention is also characterized by comprising step B′ of reacting γ-glutamylcysteine and glycine under an atmosphere having a lower oxygen concentration than atmospheric air to produce glutathione. Step B′ may be a step based on an enzymatic reaction or a step based on a chemical reaction using no enzyme. Step B′ is preferably a step based on an enzymatic reaction and is particularly preferably the step B mentioned above. The enzymatic reaction is advantageous over the chemical synthesis reaction, for the reason that e.g., protection of a substrate compound with a functional group is not required, or specificity of a reaction is high.

Examples of step B′ based on a chemical reaction using no enzyme include, but not particularly limited to, a step comprising reacting γ-glutamylcysteine, in which an a-carboxyl group and an amino group in an L-glutamic acid residue are protected with appropriate protecting groups, and glycine, in which a carboxyl group is protected with an appropriate protecting group, and subjecting a carboxyl group of one γ-glutamylcysteine molecule and an amino group of one glycine molecule to dehydration condensation to form a peptide bond. In the step, if necessary, one or more protecting groups are removed after the dehydration condensation reaction. The protecting group for a carboxyl group may be a commonly known protecting group for a carboxyl group, such as a benzyl group. The protecting group for an amino group may be a commonly known protecting group for an amino group, such as t-butoxycarbonyl (Boc) group and a 9-fluorenylmethoxycarbonyl (Fmoc) group.

Step B may use, as GSH II and/or GSH F, a cell of the organism having GSH 11 and/or GSH F activity, which may be a living cell or may be a dead but undamaged cell. Alternatively, the step may use GSH II and/or GSH F present outside the cell, more specifically, a ground material of the cell of the organism. Alternatively, the step may use GSH II and/or GSH F protein, which is isolated from the cell and appropriately purified as needed. The degree of purification of the protein having GSH II and/or GSH F activity herein is not limited, and the step may use a crude protein. The definition of a “ground material” of a cell is the same as described above.

As the enzyme, step B preferably uses no living cell having GSH II and/or GSH F activity, and more preferably, uses neither a living cell nor an undamaged dead cell having GSH II and/or GSH F activity. In particular, step B preferably uses, as the enzyme, the extracellular GSH II and/or GSH F, more specifically, GSH II and/or GSH F present in a ground material of the cell or GSH II and/or GSH F protein isolated from the cell. In the case where step B uses a living cell having GSH II and/or GSH F activity, AMP is easily decomposed in the reaction system, and thus, it is difficult to efficiently conduct an ATP regeneration reaction (see, Example 4). In contrast, step B preferably uses extracellular GSH II and/or GSH F, since AMP is rarely decomposed and the ATP regeneration reaction can efficiently proceed. In the case where the reaction of step B is carried out without using a living cell, the reduction action by the living cell is absent in the reaction system. Without such reduction action, oxidation could easily proceed under an oxygen-rich atmosphere and oxidized glutathione could be produced. However, in the present invention, the reaction of step B is carried out under an atmosphere having a lower oxygen concentration than atmospheric air. Owing to this, oxidation of glutathione is suppressed and reduced glutathione can be produced at a high yield. In an embodiment in which step B of the present invention uses extracellular GSH II and/or GSH F, more specifically, a ground material of a cell or isolated protein from the cell having GSH II and/or GSH F activity, decomposition of AMP and oxidization of glutathione can be simultaneously suppressed.

γ-Glutamylcysteine, glycine and adenosine triphosphate (ATP) in step B and γ-glutamylcysteine and glycine in step B′ can be added in the reaction system in any form, such as a salt, free from or a solvate (e.g., a hydrate).

It is preferable that γ-glutamylcysteine used as a starting material in step B and/or step B′ does not substantially contain an oxidized γ-glutamylcysteine. More specifically, γ-glutamylcysteine is contained in a percentage of 70 mol % or more relative to the total molar amount of γ-glutamylcysteine and oxidized γ-glutamylcysteine, more preferably 80 mol % or more, more preferably 90 mol % or more, further preferably 95 mol % or more, further preferably 98 mol % or more, and most preferably 100 mol %.

In step B and/or step B′, it is characterized in that the reaction is carried out under “an atmosphere having a lower oxygen concentration than atmospheric air”. As the atmosphere, an atmosphere having an oxygen concentration of 10 vol % or less is preferable, and an atmosphere having an oxygen concentration of 5 vol % or less is further preferable. The lower limit is not particularly limited and the oxygen concentration may be 0 vol %. The “atmosphere where an oxygen concentration is lower than atmospheric air” is, for example, an inert gas atmosphere. The inert gas is not particularly limited as long as oxygen is not contained therein; however, an atmosphere of an inert gas, such as nitrogen, a noble gas (e.g., argon) and carbon dioxide gas, is preferable. The phrase “oxygen is not contained therein” in the specification also includes the case where substantially no oxygen is contained. A reaction in the atmosphere satisfying the above oxygen concentration can be realized by carrying out a reaction in a reaction vessel in which the gaseous phase is replaced with the inert gas as mentioned above. The phrase “ carrying out a reaction in a reaction vessel in which the gaseous phase is replaced with the inert gas as mentioned above” also includes carrying out a reaction while supplying the inert gas flow as needed. The pressure of the atmosphere is not particularly limited and the atmospheric pressure is usually about normal pressure and can be typically 0.08 to 0.12 MPa. If glutathione is produced under such an atmosphere, production of a by-product, i.e., oxidized glutathione, is suppressed and glutathione can be efficiently produced. In step B and/or step B′, the yield of glutathione relative to a substrate, γ-glutamylcysteine, is typically 80 mol % or more, preferably 85 mol % or more, more preferably 90 mol % or more and particularly preferably 95 mol % or more. After completion of the reaction in step B and/or step B′, oxidized glutathione is not substantially contained in the reaction system (for example, the reaction mixture). Specifically, the content of glutathione relative to the total molar amount of glutathione and oxidized glutathione in the reaction system after completion of the reaction in step B and/or step B′, is 80 mol % or more, more preferably 90 mol % or more, further preferably 95 mol % or more, further preferably 97 mol % or more and most preferably 100 mol %.

In step B and/or step B′, the reaction can be carried out in a reaction mixture containing a solvent, such as water, controlled at appropriate pH. The conditions at this time are not particularly limited; however, a substrate concentration (total concentration of γ-glutamylcysteine and glycine) can be set to be preferably about 0.1 to 99 wt % and more preferably 1 to 20 wt %. At the starting time of the reaction, the quantitative ratio (molar ratio) of γ-glutamylcysteine and glycine in the substrate can be about 1 mole of glycine to 1 mole of γ-glutamylcysteine, for example, the amount of glycine relative to 1 mole of γ-glutamylcysteine can be 0.5 to 2 moles, and preferably 0.7 to 1.3 moles. The reaction temperature can be preferably 10 to 60° C. and more preferably 20 to 50° C. The pH of the reaction mixture is preferably 4 to 11 and more preferably 6 to 9. The reaction time can be preferably 1 to 120 hours and more preferably 1 to 72 hours.

Starting γ-glutamylcysteine in step B and/or step B′ can be produced in the above step A and/or step A′. In this case, glutathione can be produced from a starting material, L-cysteine, in step A and/or step A′, while suppressing production of by-products, i.e., oxidized -γ-glutamylcysteine and oxidized glutathione. The yield of glutathione relative to the substrate, L-cysteine, can be typically 75 mol % or more, preferably 80 mol % or more, more preferably 85 mol % or more, and particularly preferably 90 mol % or more.

In the case where γ-glutamylcysteine obtained in step A is used as the starting material in step B, the reaction in step A and the reaction in step B can be sequentially carried out. In this case, after completion of step A, γ-glutamylcysteine may be separated from the reaction mixture and then subjected to step B. Alternatively, step A is carried out in the conditions deficient in at least one of the elements, i.e., GSH II and/or GSH F and glycine, in other words, in the conditions in which step B cannot proceed, and then, step B may be started by supplementing the deficient element, to the reaction mixture without separating γ-glutamylcysteine from the reaction mixture after completion of step A.

The reactions of step A and step B are not necessarily carried out in order and may be simultaneously carried out. More specifically, a starting mixture containing L-cysteine, L-glutamic acid and glycine may be reacted in the presence of the enzymes used in step A and the enzyme used in step B and ATP. This embodiment is also one of the embodiments of the present invention in which oxidized γ-glutamylcysteine used as a starting material in step B, is produced by step A. However, it is known that GSH I received feedback inhibition by glutathione. In the case where step A uses GSH I as an enzyme, it is preferable that the reaction in step A and the reaction in step B are sequentially carried out. In the case where steps A and B use GSH F as an enzyme, steps A and B can be simultaneously carried out by the action of the GSH F only.

Additionally, γ-glutamylcysteine serving as a starting material in step B′ can be produced in step A′. γ-glutamylcysteine may be separated from the reaction mixture after completion of step A′ and used in step B′. After completion of step A′, step B′ may be carried out without separating γ-glutamylcysteine by adding glycine to the reaction mixture of step A′ and appropriately controlling reaction conditions. The reactions of step A′ and step B′ are not necessarily carried out in order and may be simultaneously carried out. More specifically, a starting mixture containing L-cysteine, L-glutamic acid and glycine may be reacted. This embodiment is also one of the embodiments of the present invention in which γ-glutamylcysteine serving as a starting material in step B′ is produced by step A′.

The concentration of each of the enzymes in a reaction mixture can be appropriately controlled. For example, the lower limit thereof in terms of protein is 1 μg/ml or more and the upper limit is not specified; however, it can be appropriately controlled in the range up to preferably 100 mg/ml or less. When step B uses GSH II, the GSH II activity in the reaction mixture in step B is not limited; however, the lower limit thereof is preferably 0.05 U/ml or more and the upper limit, which is not specified, can be set at usually 5000 U/ml or less. In the case where step B uses GSH F, the GSH F activity in the reaction mixture of step B is not limited; however, the lower limit can be preferably 0.05 U/ml or more and the upper limit, which is not particularly specified, can be usually 5000 U/ml or less.

The concentration of ATP in the reaction mixture can be appropriately controlled depending upon the concentration of γ-glutamylcysteine serving as a substrate and the presence or absence of the ATP regenerating system. In step B, ATP is consumed in the molar amount equal to γ-glutamylcysteine. When step B is carried out in conjugation with the ATP regeneration reaction, the addition amount of ATP relative to γ-glutamylcysteine can be greatly reduced. Then, the upper limit of the ATP concentration in the reaction mixture in step B, which is not limited, is preferably 2 fold or less and more preferably 1.2 fold or less as large as the molar concentration of γ-glutamylcysteine. The lower limit of concentration of ATP in the reaction mixture in step B, which is not limited, is preferably 0.0001 fold or more, more preferably 0.001 fold or more and further preferably 0.01 fold or more as large as the molar concentration of γ-glutamylcysteine.

### <ATP Regeneration Reaction>

Steps A and B each are a step in which ATP is consumed to produce ADP. Since ATP is a relatively expensive material, step A and/or step B are preferably carried out in conjugation with an ATP regeneration reaction for regenerating ATP from ADP produced in the steps.

As the ATP regeneration reaction, a reaction for regenerating ATP from ADP using a phosphate group supply source and phosphotransferase is mentioned.

The scheme below shows an example of ATP regeneration reaction, which uses a condensed phosphoric acid (polyphosphoric acid) as a phosphate group supply source and a combination of polyphosphate-dependent AMP transferase (PAP) and adenylate kinase (ADK) as a phosphotransferase.

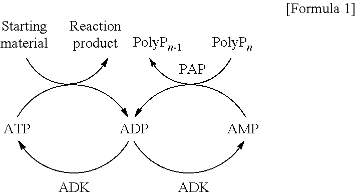

In the scheme, AMP represents adenosine monophosphate, PAP a polyphosphate-dependent AMP transferase, ADK an adenylate kinase, PolyPₙ a condensed phosphoric acid (referred to as “polyphosphoric acid” in the specification), in which “n” represents the number of phosphorus atoms, PolyPₙ₋₁ a condensed phosphoric acid (polyphosphoric acid) in which “n−1” represents the number of phosphorus atoms, “starting material” a starting material for the reaction in step A or B and “reaction product” a reaction product in step A or B.

More specifically, when step A and/or step B is carried out in the presence of a condensed phosphoric acid (polyphosphoric acid), PAP and ADK, ATP is consumed to produce ADP; ADP is then converted into ATP and AMP by the action of ADK; and AMP produced by the action of ADK is converted into ADP by the action of PAP. This ATP regeneration reaction can be carried out in conjugation with the reactions of step A and/or step B.

This reaction may use, as ADK and PAP, a cell of an organism having each of the activities of these enzymes, which may be a living cell or may be a dead but undamaged cell. Alternatively, the reaction may use ADK and/or PAP present outside the cell, more specifically, a ground material of the cell of the organism. Alternatively, the reaction may use ADK and/or PAP protein, which is isolated from the cell and appropriately purified as needed. Herein, the degree of purification of a protein having ADK and/or PAP activity is not limited, and the reaction may use ADK and/or PAP roughly purified. The “ground material” of a cell is the same as defined above.

As the enzyme, the ATP regeneration reaction, preferably, uses no living cell having ADK and/or PAP activity, and more preferably, uses neither a living cell nor an undamaged dead cell having ADK and/or PAP activity. As the enzyme to be used in step B, in particular, the ATP regeneration reaction preferably uses extracellular ADK and/or PAP, more specifically, ADK and/or PAP present in a ground material of the cell or ADK and/or PAP protein isolated from the cell. In the case where the ATP regeneration reaction uses a living cell having ADK and/or PAP activity, AMP is easily decomposed, and thus, it is difficult to efficiently conduct an ATP regeneration reaction (see Example 4). In contrast, the ATP regeneration reaction preferably uses extracellular ADK and/or PAP, since AMP is rarely decomposed and the ATP regeneration reaction can efficiently proceed. In the case where step A and/or step B is carried out without using a living cell, the reduction action by a living cell is absent in the reaction system. Without such reduction action, oxidation could easily proceed under an oxygen rich atmosphere and oxidized γ-glutamylcysteine and/or oxidized glutathione could be produced. However, in the present invention, the reaction in step A and/or step B is carried out under an atmosphere having a lower oxygen concentration than atmospheric air. Owing to this, oxidation of γ-glutamylcysteine and/or glutathione is suppressed and reduced γ-glutamylcysteine and/or glutathione can be produced at a high yield. In an embodiment in which step A and/or step B of the present invention uses extracellular ADK and/or PAP, specifically, a ground material of a cell or isolated protein from the cell having the ADK and/or PAP activity, decomposition of AMP and oxidation of γ-glutamylcysteine and/or glutathione can be simultaneously suppressed.

The concentration of each of the enzymes in the reaction mixture used in the ATP regeneration reaction can be appropriately controlled. For example, the lower limit thereof in terms of protein is 1 μg/ml or more and the upper limit is not specified; however, it can be appropriately controlled in the range up to preferably 100 mg/m or less. When step A or B is carried out in conjugation with the ATP regeneration reaction, the ADK activity in the reaction mixture is not limited; however, the lower limit thereof is preferably 2 U/ml or more. The upper limit is not specified; however, it can be usually set to be 200000 U/ml or less. The PAP activity in the reaction mixture is not particularly limited; however, the lower limit thereof is preferably 0.5 U/ml or more. The upper limit is not particularly specified, however, the upper limit can be usually set to be 50000 U/ml or less.

The addition amount of condensed phosphoric acid (polyphosphoric acid) may be appropriately controlled in accordance with the amount of substrate in the reaction. The condensed phosphoric acid (polyphosphoric acid) can be added in various forms, such as a salt (e.g., sodium salt, potassium salt), free from and a solvate (e.g., a hydrate). The degree of polymerization (the number of phosphorus atoms per molecule) of the condensed phosphoric acid (polyphosphoric acid) is not limited. Sodium metaphosphate used in Examples and Comparative Examples was a mixture of various condensed sodium phosphates different in degree of polymerization.

### EXAMPLES

### <Experiment 1>

Preparation of γ-glutamylcysteine Synthetase (GSH I) Derived from Escherichia coli K12 Strain

A DNA primer (Primer-1: SEQ ID NO: 2) having a restriction enzyme Sad cleavage site and an SD sequence bound to the nucleotide sequence corresponding to the N terminal portion of the GSH I gene (SEQ ID NO: 1); and a DNA primer (Primer-2: SEQ ID NO: 3) having a restriction enzyme KpnI cleavage site bound to the nucleotide sequence corresponding to the C terminal portion of the GSH I gene (SEQ ID NO: 1) were prepared. Using these DNA primers, DNA between these sequences was amplified by PCR to obtain a DNA fragment containing a full length of the GSH I gene. The template used in the PCR amplification was the genomic DNA of Escherichia coli K12 strain. The result of analysis of the nucleotide sequence of the obtained DNA fragment showed that the full length of GSH I gene (SEQ ID NO: 1) was contained. The obtained DNA fragment was inserted between a Sad recognition site and a KpnI recognition site present at the downstream of a lac promoter of plasmid pUC18 (GenBank Accession No. L09136, manufactured by Takara Bio Inc.) to construct a recombinant vector, pUCGSHI. E. coli HB101 competent cell (manufactured by Takara Bio Inc.) was transformed with the recombinant vector pUCGSHI to obtain E. coli HB101 (pUCGSHI). The obtained transformant was inoculated on 50 ml of 2×YT culture medium (tryptone 1.6%, yeast extract 1.0%, NaCl 0.5%, pH7.0) containing 200 μg/ml ampicillin and subjected to shaking culture at 37° C. for 24 hours. Then, enzyme activity was measured. As a result, GSH I activity was 5 U/ml and the activity of ADK derived from Escherichia coli used as a host cell was 90 U/ml. Subsequently, bacterial cells were centrifugally collected, suspended in 2.5 ml of a 100 mM phosphate buffer (pH7.0) and sonicated to obtain an enzyme solution.

### <Experiment 2>

Preparation of Glutathione Synthetase (GSH II) Derived from Escherichia coli K12 Strain

A DNA primer (Primer-3: SEQ ID NO: 5) having a restriction enzyme NdeI cleavage site bound to the nucleotide sequence corresponding to the N terminal portion of a GSH II gene (SEQ ID NO: 4); and a DNA primer (Primer-4: SEQ ID NO: 6) having a restriction enzyme EcoRI cleavage site bound to the nucleotide sequence corresponding to the C terminal portion of GSH II gene (SEQ ID NO: 4) were prepared. Using these DNA primers, DNA between these sequences was amplified by PCR to obtain a DNA fragment containing a full length of the GSH II gene. The template used in the PCR amplification was the genomic DNA of Escherichia coli K12 strain. The result of analysis of the nucleotide sequence of the obtained DNA fragment showed that the full length of GSH II gene (SEQ ID NO: 4) was contained. The obtained DNA fragment was inserted between a NdeI recognition site and an EcoRI recognition site present at the downstream of a lac promoter of plasmid pUCN18 to construct a recombinant vector, pNGSHII. The plasmid pUCN18 was a plasmid obtained by substituting “T” at the 185th positions of pUC18 (GenBank Accession No. L09136 manufactured by Takara Bio Inc.) with “A” to destruct an NdeI site and further substituting “GC” at the 471-472nd positions with “TG” to newly introduce a NdeI site. E. coli HB101 competent cell (manufactured by Takara Bio Inc.) was transformed with the recombinant vector pNGSHII to obtain E. coli HB 101 (pNGSHII). The obtained transformant was inoculated on 50 ml of 2×YT culture medium (tryptone 1.6%, yeast extract 1.0%, NaCl 0.5%, pH7.0) containing 200 μg/ml ampicillin and subjected to shaking culture at 37° C. for 24 hours. Then, enzyme activity was measured. As a result, GSH II activity was 5 U/ml and the activity of ADK derived from Escherichia coli used as a host cell was 90 U/ml. Subsequently, bacterial cells were centrifugally collected, suspended in 2.5 ml of a 100 mM phosphate buffer (pH7.0) and sonicated to obtain an enzyme solution.

<Experiment 3>

Preparation of Bifunctional Glutathione Synthetase (GSH F) Derived from Streptococcus agalactiae

A GSH F gene fragment (SEQ ID NO: 7) derived from Streptococcus agalactiae was manufactured in accordance with a gene synthesis method (by Eurogentec). In this GSH F gene fragment, the codons were optimized for expression in Escherichia coli; and a restriction enzyme NdeI cleavage site was bound to the nucleotide sequence corresponding to the N terminal portion; and a restriction enzyme EcoRI cleavage site was bound to the nucleotide sequence corresponding to the C terminal portion. The obtained DNA fragment was inserted between a NdeI recognition site and an EcoRI recognition site present at the downstream of a lac promoter of plasmid pUCN18 to construct a recombinant vector, pNGSHF. The plasmid pUCN18 was a plasmid obtained by substituting “T” at the 185th position of pUC18 (GenBank Accession No. L09136 manufactured by Takara Bio Inc.) with “A” to destruct an NdeI site and further substituting “GC” at the 471-472nd positions with “TG” to newly introduce a NdeI site. E. coli HB101 competent cell (manufactured by Takara Bio Inc.) was transformed with the recombinant vector pNGSHF to obtain E. coli HB101 (pNGSHF). The obtained transformant was inoculated on 50 ml of 2×YT culture medium (tryptone 1.6%, yeast extract 1.0%, NaCl 0.5%, pH7.0) containing 200 μg/ml ampicillin and subjected to shaking culture at 37° C. for 24 hours. Then, enzyme activity was measured. As a result, GSH F activity was 3 U/ml and the activity of ADK derived from Escherichia coli used as a host cell was 90 U/ml. Subsequently, bacterial cells were centrifugally collected, suspended in 2.5 ml of a 100 mM phosphate buffer (pH7.0) and sonicated to obtain an enzyme solution.

### <Experiment 4>

Preparation of AMP phosphotransferase (PAP) derived from Acinetobacter johnsonii A PAP gene fragment (SEQ ID NO: 8) was manufactured in accordance with a gene synthesis method (by Eurogentec). In this PAP gene fragment, the codons were optimized for expression in Escherichia coli; and a restriction enzyme NdeI cleavage site was bound to the nucleotide sequence corresponding to the N terminal portion; and a restriction enzyme EcoRI cleavage site was bound to the nucleotide sequence corresponding to the C terminal portion. The obtained DNA fragment was inserted between a NdeI recognition site and an EcoRI recognition site present at the downstream of a lac promoter of plasmid pUCN18 to construct a recombinant vector, pNPAP. The plasmid pUCN18 was a plasmid obtained by substituting “T” at the 185th position of pUC18 (GenBank Accession No. L09136 manufactured by Takara Bio Inc.) with “A” to destruct an NdeI site and further substituting “GC” at the 471-472nd positions with “TG” to newly introduce a NdeI site. E. coli HB101 competent cell (manufactured by Takara Bio Inc.) was transformed with the recombinant vector pNPAP to obtain E. coli HB101 (pNPAP). The obtained transformant was inoculated on 50 ml of 2×YT culture medium (tryptone 1.6%, yeast extract 1.0%, NaCl 0.5%, pH7.0) containing, 200 μg/m1 ampicillin and subjected to shaking culture at 37° C. for 24 hours. Then, enzyme activity was measured. As a result, PAP activity was 40 U/ml and the activity of ADK derived from Escherichia coli used as a host cell was 90 U/ml. Subsequently, bacterial cells were centrifugally collected, suspended in 2.5 ml of a 100 mM phosphate buffer (pH7.0) and sonicated to obtain an enzyme solution.

### <Calculation of Yield>

A method of calculating the yield of each of the compounds obtained in the Experiments herein was as follows.

A reaction product was quantified based on analysis by high-performance liquid chromatography and then the yield thereof was determined by the following expression:

Yield: the production amount (mol) of compound/starting L-cysteine (mol)×100

The conditions of the above high-performance liquid chromatography are as follows. In the elution conditions, glutathione (GSH), γ-glutamylcysteine (γ-GC), oxidized γ-GC, oxidized glutathione (GSSG) sequentially elute in this order.

### [Analysis of Yield]

Column: ODS-HG-3 (4.6 mmφ×150 mm, manufactured by Nomura Chemical Co., Ltd.);

Eluent: Solution prepared by dissolving potassium dihydrogenphosphate (12.2 g) and sodium heptane sulfonate (3.6 g) in distilled water (1.8 L), controlling pH of the solution to be pH 2.8 with phosphoric acid and further adding methanol (186 ml);

Flow rate: 1.0 ml/minute;

Column temperature: 40° C.;

Measurement wavelength: 210 nm.

### Example 1

### Reaction in System Containing an Equivalent Amount of ATP Under a Nitrogen Atmosphere

The reaction of Example 1 described below was carried out under a nitrogen atmosphere.

(Production of γ-glutamylcysteine)

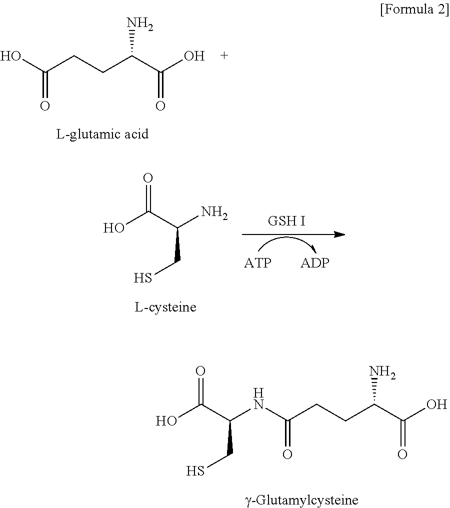 [SMILES: N[C@@H](CCC(=O)O)C(=O)O ; N[C@@H](CS)C(=O)O ; N[C@@H](CCC(=O)N[C@@H](CS)C(=O)O)C(=O)O]

Monosodium L-glutamate monohydrate (0.3668 g (2.17 mmol)), L-cysteine hydrochloride monohydrate (0.3636 g (2.07 mmol)), magnesium sulfate hepta hydrate (1.02 g), ATP (1.19 g (2.16 mmol)), and distilled water (15 g) were mixed. The pH of the mixture was controlled to be 7.5 with a 15 wt % aqueous sodium hydroxide solution (1.4 g). To the mixture, the γ-glutamylcysteine synthetase (GSH I) solution (1 g) prepared in Experiment 1 was added and a reaction was initiated. The reaction was carried out in a reaction vessel provided with a nitrogen port and an exhaust port in the conditions where the oxygen concentration of the gaseous phase was maintained at 0 vol % as much as possible by supplying nitrogen at a rate of 10 ml/min through the nitrogen port to purge the air (gaseous phase) within the reaction vessel. The temperature during the reaction was set at 30° C. The reaction continuously proceeded and produced γ-glutamylcysteine and oxidized glutamylcysteine. Six hours after initiation of the reaction, L-cysteine disappeared. The yields of γ-glutamylcysteine, and oxidized glutamylcysteine six hours after initiation of the reaction relative to the starting L-cysteine were 97 mol % and 1 mol %, respectively.

(Production of Glutathione)

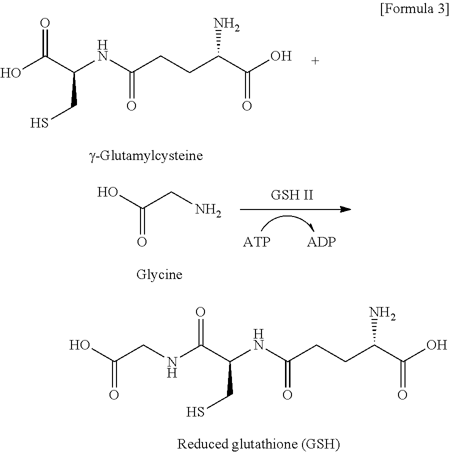 [SMILES: N[C@@H](CCC(=O)N[C@@H](CS)C(=O)O)C(=O)O ; NCC(=O)O ; N[C@@H](CCC(=O)N[C@@H](CS)C(=O)NCC(=O)O)C(=O)O]

After the 6-hour reaction, to the reaction solution, glycine (0.192 g (2.56 mmol)), magnesium sulfate hepta hydrate (1.02 g) and ATP (1.19 g (2.16 mmol)) were mixed and the pH of the mixture was controlled to be 7.5 with a 15 wt % aqueous sodium hydroxide solution (1.4 g). To the reaction mixture, the glutathione synthetase (GSH II) solution (1 g) prepared in Experiment 2 was added and a reaction was initiated. The reaction was carried out under a nitrogen atmosphere in the same manner as in the step of producing γ-glutamylcysteine. The temperature during the reaction was set at 30° C. The reaction continuously proceeded. Four hours after initiation of the reaction, γ-glutamylcysteine and oxidized glutamylcysteine disappeared and glutathione and oxidized glutathione were produced. The yields of glutathione and oxidized glutathione four hours after initiation of the reaction relative to the starting L-cysteine were 94 mol % and 2 mol %, respectively.

### Example 2

### Reaction with ATP Regenerating System Under a Nitrogen Atmosphere

The reaction of Example 2 described below was carried out under a nitrogen atmosphere.

(Production of γ-glutamylcysteine)

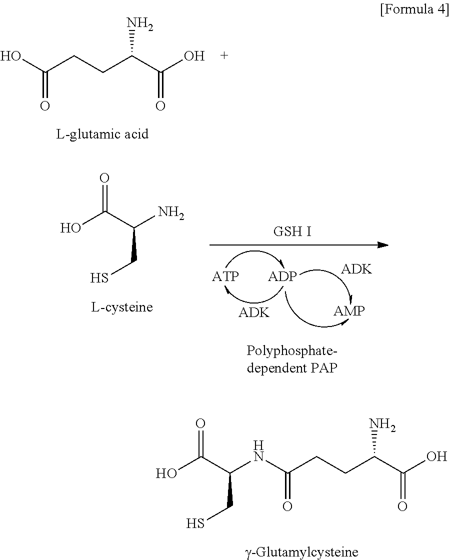 [SMILES: N[C@@H](CCC(=O)O)C(=O)O ; N[C@@H](CS)C(=O)O ; "ADK" ; "ADK" ; N[C@@H](CCC(=O)N[C@@H](CS)C(=O)O)C(=O)O]

Monosodium L-glutamate monohydrate (0.389 g (2.30 mmol)), L-cysteine hydrochloride monohydrate (0.3714 g (2.11 mmol)), magnesium sulfate hepta hydrate (0.7068 g), ATP (0.0588 g (0.11 mmol)), sodium metaphosphate (0.8 g) and distilled water (14 g) were mixed and the pH of the mixture was controlled to be 7.5 with a 15 wt % aqueous sodium hydroxide solution (0.8 g). To the mixture, the γ-glutamylcysteine synthetase (GSH I) solution (1 g) prepared in Experiment 1 and the PAP solution (1 g) prepared in Experiment 4 were added and a reaction was initiated. The reaction was carried out in a reaction vessel provided with a nitrogen port and an exhaust port in the conditions where the oxygen concentration of the gaseous phase was maintained at 0 vol % as much as possible by supplying nitrogen at a rate of 10 ml/min through the nitrogen port to purge the air (gaseous phase) within the reaction vessel. The temperature during the reaction was set at 30° C. The reaction continuously proceeded and produced γ-glutamylcysteine and oxidized glutamylcysteine. Six hours after initiation of the reaction, L-cysteine disappeared. The yields of γ-glutamylcysteine and oxidized glutamylcysteine six hours after initiation of the reaction relative to the starting L-cysteine were 95 mol % and 1 mol %, respectively.

(Production of Glutathione)

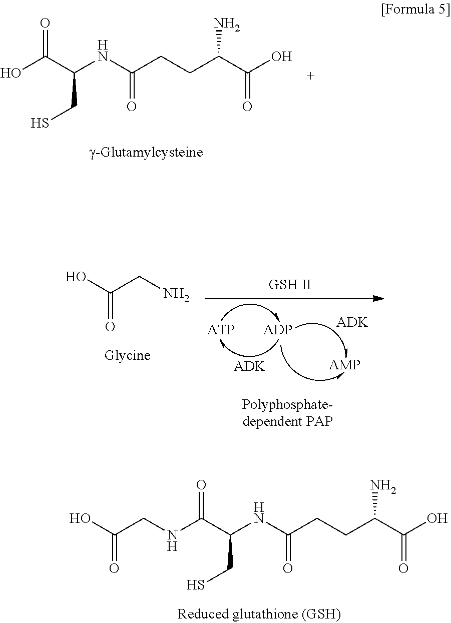 [SMILES: N[C@@H](CCC(=O)N[C@@H](CS)C(=O)O)C(=O)O ; NCC(=O)O ; "ADK" ; "ADK" ; N[C@@H](CCC(=O)N[C@@H](CS)C(=O)NCC(=O)O)C(=O)O]

After the 6-hour reaction, to the reaction solution, glycine (0.19 g (2.53 mmol)) was added and the pH of the mixture was controlled to be 7.5 with a 15 wt % aqueous sodium hydroxide solution (0.2 g). To the reaction mixture, the glutathione synthetase (GSH II) solution (1 g) prepared in Experiment 2 and the PAP solution (1 g) prepared in Experiment 4 were added and a reaction was initiated. The reaction was carried out under a nitrogen atmosphere in the same manner as in the step of producing γ-glutamylcysteine. The temperature during the reaction was set at 30° C. The reaction continuously proceeded and produced glutathione and oxidized glutathione. Six hours after initiation of the reaction, γ-glutamylcysteine and oxidized glutamylcysteine disappeared. The yields of glutathione and oxidized glutathione six hours after initiation of the reaction relative to the starting L-cysteine were 82 mol % and 2 mol %, respectively.

Since the GSH I solution prepared in Experiment 1, GSH II solution prepared in Experiment 2 and PAP solution prepared in Experiment 4 had ADK activity derived from Escherichia coli used as a host cell, no separate preparation of an ADK solution was required in the aforementioned two steps.

### Example 3

### Reaction with ATP Regenerating System Under a Nitrogen Atmosphere

The reaction of the Example described below was carried out under a nitrogen atmosphere.

(Production of Glutathione)

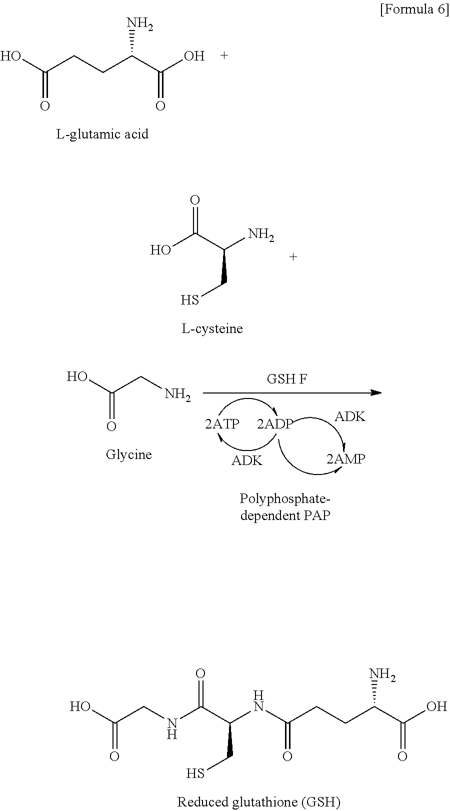 [SMILES: N[C@@H](CCC(=O)O)C(=O)O ; N[C@@H](CS)C(=O)O ; NCC(=O)O ; "ADK" ; "ADK" ; N[C@@H](CCC(=O)N[C@@H](CS)C(=O)NCC(=O)O)C(=O)O]

Monosodium L-glutamate monohydrate (0.185 g (1.09 mmol)), L-cysteine hydrochloride monohydrate (0.175 g (1.00 mmol)), glycine (1.09 mmol), magnesium sulfate hepta hydrate (0.35 g), ATP (0.055 g (0.10 mmol)), sodium metaphosphate (0.4 g) and distilled water (16 g) were mixed. The pH of the mixture was controlled to be 7.5 with a 15 wt % aqueous sodium hydroxide solution (0.44 g). To the mixture, the bifunctional glutathione synthetase (GSH F) solution (1 g) prepared in Experiment 3 and the PAP solution (1 g) prepared in Experiment 4 were added and a reaction was initiated. The reaction was carried out in a reaction vessel provided with a nitrogen port and an exhaust port in the conditions where the oxygen concentration of the gaseous phase was maintained at 0 vol % as much as possible by supplying nitrogen at a rate of 10 ml/min through the nitrogen port to purge the air (gaseous phase) within the reaction vessel. The temperature during the reaction was set at 30° C. The reaction continuously proceeded and produced glutathione and oxidized glutathione. Six hours after initiation of the reaction, L-cysteine disappeared. The yields of glutathione and oxidized glutathione six hours after initiation of the reaction relative to the starting L-cysteine were 90 mol % and 1 mol %, respectively.

Since the GSH F solution prepared in Experiment 3 and the PAP solution prepared in Experiment 4 had ADK activity derived from Escherichia coli used as a host cell, no separate preparation of an ADK solution was required in the step mentioned above.

### Comparative Example 1

### Reaction in System Containing an Equivalent Amount of ATP Under an Air Atmosphere>

The reaction of Comparative Example 1 described below was carried out under an air atmosphere.

(Production of γ-glutamylcysteine)

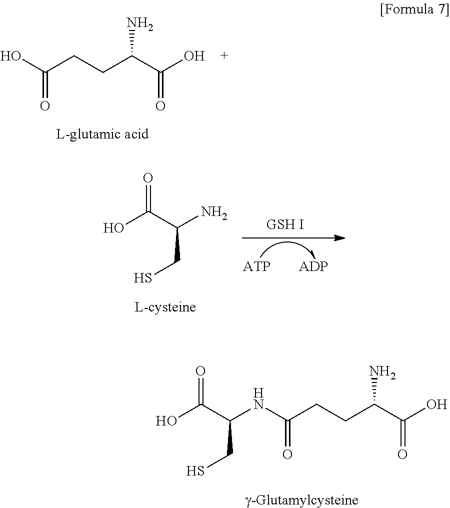 [SMILES: N[C@@H](CCC(=O)O)C(=O)O ; N[C@@H](CS)C(=O)O ; N[C@@H](CCC(=O)N[C@@H](CS)C(=O)O)C(=O)O]

Monosodium L-glutamate monohydrate (0.3668 g (2.17 mmol)), L-cysteine hydrochloride monohydrate (0.3636 g (2.07 mmol)), magnesium sulfate hepta hydrate (1.02 g), ATP (1.19 g (2.16 mmol)), and distilled water (15 g) were mixed. The pH of the mixture was controlled to be 7.5 with a 15 wt % aqueous sodium hydroxide solution (1.4 g). To the mixture, the γ-glutamylcysteine synthetase (GSH I) solution (1 g) prepared in Experiment 1 was added and a reaction was initiated. The reaction was carried out in the conditions where the reaction solution was in contact with the air within a reaction vessel without replacement with nitrogen. The temperature during the reaction was set at 30° C. The reaction continuously proceeded and produced γ-glutamylcysteine and oxidized glutamylcysteine. Six hours after initiation of the reaction, L-cysteine disappeared. The yields of γ-glutamylcysteine and oxidized glutamylcysteine six hours after initiation of the reaction relative to the starting L-cysteine were 78 mol % and 10 mol %, respectively.

(Production of Glutathione)

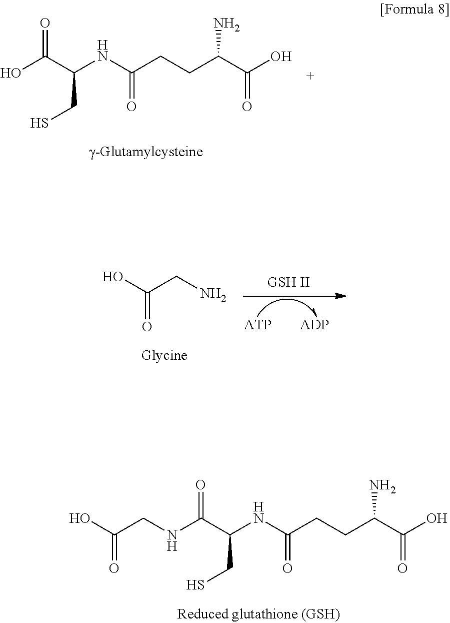 [SMILES: N[C@@H](CCC(=O)N[C@@H](CS)C(=O)O)C(=O)O ; NCC(=O)O ; N[C@@H](CCC(=O)N[C@@H](CS)C(=O)NCC(=O)O)C(=O)O]

After the 6-hour reaction, to the reaction solution, glycine (0.192 g (2.56 mmol)), magnesium sulfate hepta hydrate (1.02 g) and ATP (1.19 g (2.16 mmol)) were added and the pH of the mixture to be 7.5 with a 15 wt % aqueous sodium hydroxide solution (1.4 g). To the reaction mixture, the glutathione synthetase (GSH II) solution (1 g) prepared in Experiment 2 was added and a reaction was initiated. The reaction was carried out in the conditions where the reaction solution was in contact with the air within a reaction vessel without replacement with nitrogen. The temperature during the reaction was set at 30° C. The reaction continuously proceeded. Four hours after initiation of the reaction, γ-glutamylcysteine and oxidized glutamylcysteine disappeared and glutathione and oxidized glutathione were produced. The yields of glutathione and oxidized glutathione four hours after initiation of the reaction relative to the starting L-cysteine were 58 mol % and 20 mol %, respectively.

### Comparative Example 2

### Reaction with ATP Regenerating System Under an Air Atmosphere

The reaction of Comparative Example 2 described below was carried out under an air atmosphere.

(Production of γ-glutamylcysteine)

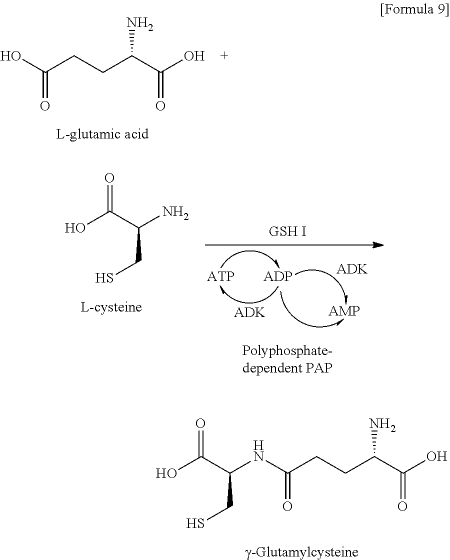 [SMILES: N[C@@H](CCC(=O)O)C(=O)O ; N[C@@H](CS)C(=O)O ; "ADK" ; "ADK" ; N[C@@H](CCC(=O)N[C@@H](CS)C(=O)O)C(=O)O]

Monosodium L-glutamate monohydrate (0.389 g (2.30 mmol)), L-cysteine hydrochloride monohydrate (0.3714 g (2.11 mmol)), magnesium sulfate hepta hydrate (0.7068 g), ATP (0.0588 g (0.11 mmol)), sodium metaphosphate (0.8 g) and distilled water (14 g) were mixed. The pH of the mixture was controlled to be 7.5 with a 15 wt % aqueous sodium hydroxide solution (0.8 g). To the mixture, the γ-glutamylcysteine synthetase (GSH I) solution (1 g) prepared in Experiment 1 and the PAP solution (1 g) prepared in Experiment 4 were added and a reaction was initiated. The reaction was carried out in the conditions where the reaction solution was in contact with the air within a reaction vessel without replacement with nitrogen. The temperature during the reaction was set at 30° C. The reaction continuously proceeded and produced γ-glutamylcysteine and oxidized glutamylcysteine. Six hours after initiation of the reaction, L-cysteine disappeared. The yields of γ-glutamylcysteine and oxidized glutamylcysteine six hours after initiation of the reaction relative to the starting L-cysteine were 72 mol % and 13 mol %, respectively.

(Production of Glutathione)

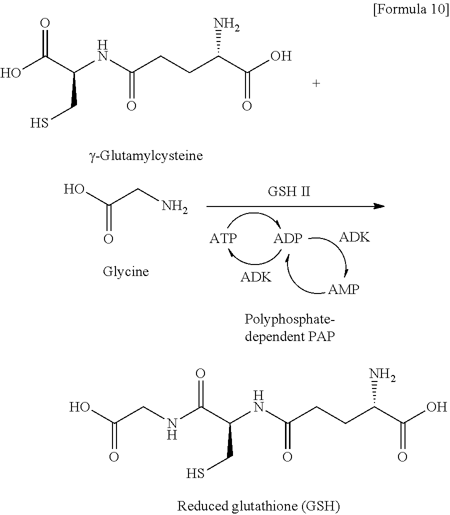 [SMILES: N[C@@H](CCC(=O)N[C@@H](CS)C(=O)O)C(=O)O ; NCC(=O)O ; "ADK" ; "ADK" ; N[C@@H](CCC(=O)N[C@@H](CS)C(=O)NCC(=O)O)C(=O)O]

After the 6-hour reaction, to the reaction solution, glycine (0.19 g (2.53 mmol)) was added and the pH of the mixture was controlled to be 7.5 with a 15 wt % aqueous sodium hydroxide solution (0.2 g). To the reaction mixture, the glutathione synthetase (GSH II) solution (1 g) prepared in Experiment 2 and the PAP solution (1 g) prepared in Experiment 4 were added and a reaction was initiated. The reaction was carried out in the conditions where the reaction solution was in contact with the air within a reaction vessel without replacement with nitrogen. The temperature during the reaction was set at 30° C. The reaction continuously proceeded and produced glutathione and oxidized glutathione. Six hours after initiation of the reaction, γ-glutamylcysteine and oxidized γ-glutamylcysteine disappeared. The yields of glutathione and oxidized glutathione six hours after initiation of the reaction relative to the starting L-cysteine were 51 mol % and 22 mol %, respectively.

Since the GSH I solution prepared in Experiment 1, GSH II solution prepared in Experiment 2 and PAP solution prepared in Experiment 4 had ADK activity derived from Escherichia coli used as a host cell, no separate preparation of an ADK solution was required in the aforementioned two steps.

### Comparative Example 3

### Reaction with ATP Regenerating System Under an Air Atmosphere

The reaction of the Comparative Example described below was carried out under an air atmosphere.

(Production of Glutathione)

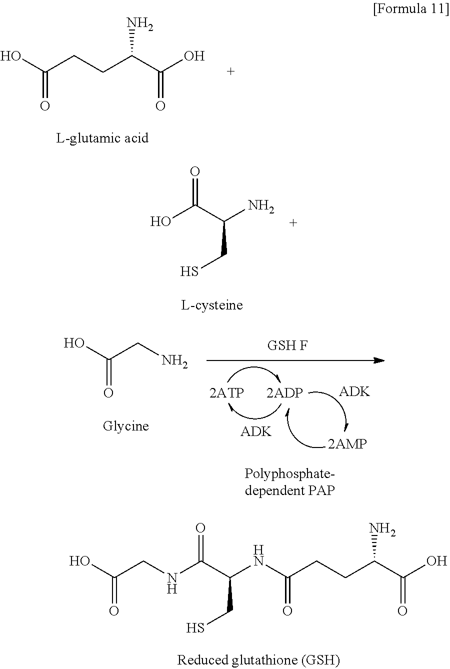 [SMILES: N[C@@H](CCC(=O)O)C(=O)O ; N[C@@H](CS)C(=O)O ; NCC(=O)O ; "ADK" ; "ADK" ; N[C@@H](CCC(=O)N[C@@H](CS)C(=O)NCC(=O)O)C(=O)O]

Monosodium L-glutamate monohydrate (0.185 g (1.09 mmol)), L-cysteine hydrochloride monohydrate (0.175 g (1.00 mmol)), glycine (1.09 mmol), magnesium sulfate hepta hydrate (0.35 g), ATP (0.055 g (0.10 mmol)), sodium metaphosphate (0.4 g) and distilled water (16 g) were mixed. The pH of the mixture was controlled to be 7.5 with a 15 wt % aqueous sodium hydroxide solution (0.44 g). To the mixture, the bifunctional glutathione synthetase (GSH F) solution (1 g) prepared in Experiment 3 and the PAP solution (1 g) prepared in Experiment 4 were added and a reaction was initiated. The reaction was carried out in the conditions where the reaction solution was in contact with the air within a reaction vessel without replacement with nitrogen. The temperature during the reaction was set at 30° C. The reaction continuously proceeded and produced glutathione and oxidized glutathione. Six hours after initiation of the reaction, L-cysteine disappeared. The yields of glutathione and oxidized glutathione six hours after initiation of the reaction relative to the starting L-cysteine were 36 mol % and 14 mol %, respectively. Note that oxidized γ-glutamylcysteine was produced at a yield of 6 mol %.

Since the GSH F solution prepared in Experiment 3 and the PAP solution prepared in Experiment 4 have ADK activity derived from Escherichia coli used as a host cell, no separate preparation of an ADK solution was required in the step mentioned above.

### Example 4

### Reaction with ATP Regenerating System Using Unground Cells Under a Nitrogen Atmosphere

The reaction described below was carried out under a nitrogen atmosphere.

(Production of Glutathione)

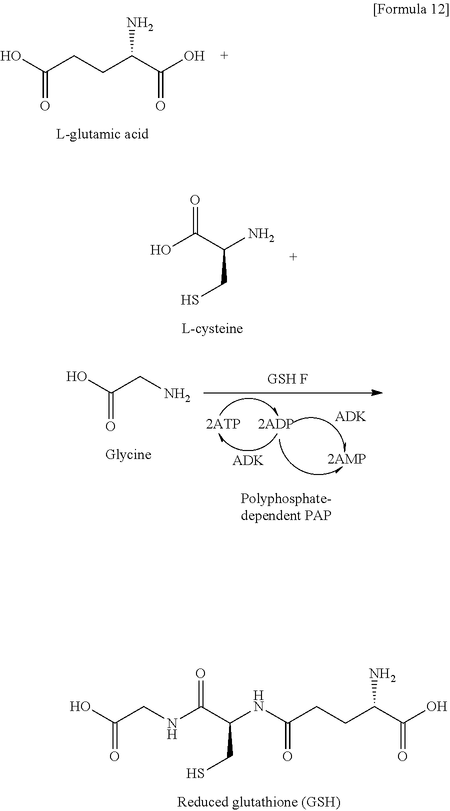 [SMILES: N[C@@H](CCC(=O)O)C(=O)O ; N[C@@H](CS)C(=O)O ; NCC(=O)O ; "ADK" ; "ADK" ; N[C@@H](CCC(=O)N[C@@H](CS)C(=O)NCC(=O)O)C(=O)O]

Monosodium L-glutamate monohydrate (0.185 g (1.09 mmol)), L-cysteine hydrochloride monohydrate (0.175 g (1.00 mmol)), glycine (1.09 mmol), magnesium sulfate hepta hydrate (0.35 g), ATP (0.055 g (0.10 mmol)), sodium metaphosphate (0.4 g) and distilled water (16 g) were mixed. The pH of the mixture was controlled to be 7.5 with a 15 wt % aqueous sodium hydroxide solution (0.44 g). To the mixture, a suspension (1 g) containing unground cells of a recombinant Escherichia coli expressing GSH F and a suspension (1 g) containing unground cells of a recombinant Escherichia coli expressing PAP were added and a reaction was initiated. The reaction was carried out in a reaction vessel provided with a nitrogen port and an exhaust port in the conditions where the oxygen concentration of the gaseous phase was maintained at 0 vol % as much as possible by supplying nitrogen at a rate of 10 ml/min through the nitrogen port to purge the air (gaseous phase) within the reaction vessel. The temperature during the reaction was set at 30° C. One hour after initiation of the reaction, the reaction mixture was analyzed. As a result, production of glutathione and oxidized glutathione was found. The conversion rates of them relative to the starting L-cysteine were 7.3 mol % and 0.6 mol %, respectively. Thereafter, the reaction proceeded and virtually stopped seven hours after initiation of the reaction. The conversion rates of them relative to the starting L-cysteine were 10.8 mol % and 1.2 mol %, respectively. ATP, ADP and AMP almost disappeared. Decomposed products of AMP, i.e., adenine, adenosine and hypoxanthine, were found.

The “suspension containing unground cells of a recombinant Escherichia coli expressing GSH F” was prepared in the same manner and using the same materials as in Example 3 except that an operation described at the end of the description of Experiment 3 by the phrase: “Subsequently, bacterial cells were centrifugally collected, suspended in 2.5 ml of a 100 mM phosphate buffer (pH7.0) and sonicated to obtain an enzyme solution” was replaced by an operation described by the phrase: “Subsequently, bacterial cells were centrifugally collected, suspended in 2.5 ml of a 100 mM phosphate buffer (pH7.0) to obtain a suspension containing unground cells of a recombinant Escherichia coli expressing GSH F”.

The “suspension containing unground cells of a recombinant Escherichia coli expressing PAP” was prepared in the same manner and using the same materials as in Example 4 except that an operation described at the end of the description of Experiment 4 by the phrase: “Subsequently, bacterial cells were centrifugally collected, suspended in 2.5 ml of a 100 mM phosphate buffer (pH7.0) and sonicated to obtain an enzyme solution” was replaced by an operation described by the phrase: “Subsequently, bacterial cells were centrifugally collected, suspended in 2.5 ml of a 100 mM phosphate buffer (pH7.0) to obtain a suspension containing unground cells of a recombinant Escherichia coli expressing PAP”.

Since the “suspension containing unground cells of a recombinant Escherichia coli expressing GSH F and the suspension containing unground cells of a recombinant Escherichia coli expressing PAP prepared by the aforementioned methods had ADK activity derived from Escherichia coli used as a host cell, no separate preparation of an ADK solution was required in the step mentioned above.

### Sequence Listing Free Text

SEQ ID NO: 2: primer

SEQ ID NO: 3: primer

SEQ ID NO: 5: primer

SEQ ID NO: 6: primer

All publications including Patents and Patent Applications cited in the specification are incorporated in the specification by reference in their entirety.

1. A method for producing γ-glutamylcysteine, comprising:
reacting L-cysteine and L-glutamic acid under an atmosphere having a lower oxygen concentration than atmospheric air to produce γ-glutamylcysteine.

2. The method according to claim 1, wherein the reacting of L-cysteine and L-glutamic acid is carried out by the action of at least one enzyme selected from the group consisting of γ-glutamylcysteine synthetase and a bifunctional glutathione synthetase in the presence of adenosine triphosphate (ATP).

3. The method according to claim 2, wherein the reacting of L-cysteine and L-glutamic acid is carried out in conjugation with an ATP regeneration reaction for regenerating adenosine diphosphate (ADP) into adenosine triphosphate (ATP).

4. The method according to claim 2, wherein the γ-glutamylcysteine synthetase is derived from Escherichia coli.

5. The method according to claim 2, wherein the bifunctional glutathione synthetase is derived from Streptococcus agalactiae.

6. A method for producing glutathione, comprising:
reacting γ-glutamylcysteine and glycine under an atmosphere having a lower oxygen concentration than atmospheric air to produce glutathione.

7. The method according to claim 6, wherein the reacting of γ-glutamylcysteine and glycine is carried out by the action of at least one enzyme selected from the group consisting of glutathione synthetase and a bifunctional glutathione synthetase in the presence of adenosine triphosphate (ATP).

8. The method according to claim 7, wherein the reacting of γ-glutamylcysteine and glycine is carried out in conjugation with an ATP regeneration reaction for regenerating adenosine diphosphate (ADP) into adenosine triphosphate (ATP).

9. The method according to claim 7, wherein the glutathione synthetase is derived from Escherichia coli.

10. The method according to claim 7, wherein the bifunctional glutathione synthetase is derived from Streptococcus agalactiae.

11. The method according to claim 6, further comprising, prior to the reacting of γ-glutamylcysteine and glycine:
reacting L-cysteine and L-glutamic acid under an atmosphere having a lower oxygen concentration than atmospheric air to produce the γ-glutamylcysteine.

12. The method according to claim 11, wherein the reacting of L-cysteine and L-glutamic acid is carried out by the action of at least one enzyme selected from the group consisting of γ-glutamylcysteine synthetase and a bifunctional glutathione synthetase in the presence of adenosine triphosphate (ATP).

13. The method according to claim 12, wherein the reacting of L-cysteine and L-glutamic acid is carried out in conjugation with an ATP regeneration reaction for regenerating adenosine diphosphate (ADP) into adenosine triphosphate (ATP).

14. The method according to claim 12, wherein the γ-glutamylcysteine synthetase is derived from Escherichia coli.

15. The method according to claim 12, wherein the bifunctional glutathione synthetase is derived from Streptococcus agalactiae.

16. The method according to claim 2, wherein the γ-glutamylcysteine synthetase has an amino acid sequence having 95% or more identity to the amino acid sequence of SEQ ID NO: 9, and the bifunctional glutathione synthetase has an amino acid sequence having 95% or more identity to the amino acid sequence of SEQ ID NO: 12.

17. The method according to claim 7, wherein the glutathione synthetase has an amino acid sequence having 95% or more identity to the amino acid sequence of SEQ ID NO: 10.

18. The method according to claim 12, wherein the reacting of γ-glutamylcysteine and glycine is carried out by the action of at least one enzyme selected from the group consisting of glutathione synthetase and a bifunctional glutathione synthetase in the presence of adenosine triphosphate (ATP), the γ-glutamylcysteine synthetase has an amino acid sequence having 95% or more identity to the amino acid sequence of SEQ ID NO: 9, the bifunctional glutathione synthetase has an amino acid sequence having 95% or more identity to the amino acid sequence of SEQ ID NO: 12, and the glutathione synthetase has an amino acid sequence having 95% or more identity to the amino acid sequence of SEQ ID NO: 10.
